(19) United States
(12) Patent Application Publication
(10) Pub. No.: US 2018/0127754 A1
(43) Pub. Date: May 10, 2018

(54) BIVALENT ANTISENSE OLIGONUCLEOTIDES

(71) Applicant: MIRRX THERAPEUTICS A/S, Lyngby (DK)

(72) Inventors: Thorleif Moeller, Limhamn (SE); Christina Udesen, Limhamn (SE)

(21) Appl. No.: 15/809,567

(22) Filed: Nov. 10, 2017

Related U.S. Application Data
(63) Continuation of application No. 13/636,459, filed on Dec. 5, 2012, now abandoned.

(30) Foreign Application Priority Data
Mar. 24, 2010 (DK) PA 2010 00241

(51) Int. Cl. C12N 15/113; C12N 15/11

(52) U.S. Cl. CPC C12N 15/113; C12N 2310/51; C12N 2310/3519; C12N 2310/331; C12N 2310/3231; C12N 2310/3183; C12N 2310/3181; C12N 2310/318; C12N 2310/113; C12N 2310/11; C12N 15/111

(57) ABSTRACT

The present invention provides bivalent molecules comprising a first oligonucleotide linked to a second oligonucleotide. The first and the second oligonucleotide are preferably linked via a linking moiety. Preferably, both the first and/or the second oligonucleotide comprise an antisense sequence complementary to a cellular RNA such as mRNA or microRNA.

### BACKGROUND

MicroRNAs are small noncoding RNA that bind to microRNA binding sites in target RNA to impose translational regulation or altered stability of the target RNA. Typically, the activity of the target RNA is decreased either because the microRNA destabilizes the target RNA to which it binds or because microRNA binding to the target RNA leads to translational repression.

Currently, it is estimated that between 500 and 1000 human microRNA exist and it is estimated that more than 50% of all human genes are subject to microRNA regulation. A specific microRNA may bind to and regulate a large number of target RNAs (typically mRNAs) e.g. up to 100 target RNA. Moreover, a specific target RNA may comprise several microRNA binding sites for identical or different microRNAs. When several microRNAs bind to the same target RNA, they often bind cooperatively.

Given the number of microRNA and also the number of genes estimated to be regulated by microRNAs, it is expected that microRNAs play a role in many, if not most gene regulatory processes and also in disease development and disease states. Indeed, it is becoming increasingly clear that microRNAs play a role in many diseases.

Therefore, there is great interest in being able to modulate microRNA regulatory pathways.

Fundamentally, two ways of negatively affecting microRNA regulatory pathways may be contemplated.

First, microRNAs may be inactivated, e.g. by molecules that bind directly to microRNAs. This approach has been used almost since microRNAs were discovered. Thus, already in 2003 steric blockers binding to microRNA was described (also termed antimirs or antagomirs). The consequence of such an approach is that all target RNAs of a given microRNA is deregulated.

A second approach was described in WO2008/061537. This approach employs so-called Blockmirs that bind to microRNA binding sites in target RNAs. Thus, Blockmirs enable specific deregulation of one specific microRNA target of a given microRNA, while allowing the microRNA to regulate all its other targets.

While Blockmirs and antimirs are very important molecules that can be used to modulate microRNA regulatory pathways, they have some shortcomings. Thus, an antimir as described in the state of the art cannot simultaneously bind to two different or identical microRNAs (or even microRNA families) which may be desirable in some situations.

Moreover, a Blockmirs as described in the prior art cannot simultaneously bind to two microRNA bindings sites, which may be desirable in some situations.

### BRIEF DESCRIPTION OF THE DRAWINGS

FIG. 1.

Dual luciferase measurements of the activity of bivalent molecules of the invention, refer to example 4 for details.

FIG. 2.

Dual luciferase measurements of the activity of bivalent molecules of the invention, refer to example 4 for details.

FIG. 3.

Dual luciferase measurements of the activity of bivalent molecules of the invention, refer to example 5 for details.

FIG. 4.

Dual luciferase measurements of the activity of bivalent molecules of the invention, refer to example 5 for details.

### DISCLOSURE OF THE INVENTION

### Definitions and Terms

When referring to the “activity of a target mRNA”, what is typically meant is the expression of the target mRNA, i.e. translation into a protein or peptide. Thus, regulation of the activity of a target mRNA may include degradation of the mRNA and/or translational regulation. The activity may also be replication.

The terms “regulate” and “modulate” are used interchangeably herein.

As used herein, regulation may be either positive or negative. I.e. a regulator (e.g. oligonucleotide or microRNA) may increase the activity of the target (e.g. target mRNA) or it may decrease the activity of the target.

When the target RNA is a viral RNA, the molecules of the invention may affect replication of the virus or otherwise interfere with the proliferation of the virus.

The term microRNA as used herein has the same meaning as typically in the art. I.e. the term microRNA refers to a small non-translated RNA of typically 18-22 nucleotides that is capable of regulating the activity of a target mRNA. A microRNA is typically processed from pri-microRNA to short stem-loop structures called pre-microRNA and finally to mature miRNA. Both strands of the stem of the pre-microRNA may be processed to a mature microRNA.

The miRBase (http://microrna.sanger.ac.uk/sequences/) is a compilation of known microRNAs. Also predicted and known targets of the microRNAs can be found on this site.

The term siRNA (short interfering RNA) as used herein has the same meaning as typically in the art. I.e. the term siRNA refers to double stranded RNA complex wherein the strands are typically 18-22 nucleotides in length. Very often, the complex has 3′-overhangs.

When referring to the RNAi machinery herein, what is meant are the cellular components necessary for the activity of siRNAs and/or microRNAs or for the RNAi pathway. A major player of the RNAi machinery is the RNA induced silencing complex (the RISC complex).

As referred to herein, an RNA unit is one of the monomers that make up an RNA polymer/oligomer. Thus, an RNA unit is also referred to as an RNA monomer or a RNA nucleotide. Likewise, a DNA unit is one of the monomers that make up a DNA polymer/oligomer and a DNA unit may also be referred to as a DNA monomer or a DNA nucleotide.

When referring to a base, what is meant is the base (also termed nucleobase) of a nucleotide. The base may be part of DNA, RNA, INA, LNA or any other nucleic acid capable of engaging in Watson Crick duplex formation and preferably in specific base pairing. The base may also be part of PNA (peptide nucleic acid) or morpholino. In some embodiments, the base may be a universal base.

When referring to the length of a sequence or oligonucleotide, reference may be made to the number of (repeating) units or to the number of bases.

When referring to a complementary sequence, G pairs to C, A pairs to T and U and vice versa. In a preferred embodiment, G also pairs to U and vice versa to form a so-called wobble base pair. In another preferred embodiment, the base inosine (I) may be substituted for A in any of SEQ ID NOs 1-723 (as may occur by A to I editing) or I may be substituted for A in sequences complementary to any of SEQ ID NOs 1-723. I basepairs to A, C and U. I may also be used in the molecules of the invention. In still another preferred embodiment, universal bases may be used in the molecules of the invention, e.g. no more than 1, 2 or 3 universal bases per molecule. Universal bases can typically basepair to G, C, A, U and T. Often universal bases do not form hydrogen bonds with the opposing base on the other strand. In still another preferred embodiment, a complementary sequence refers to a contiguous sequence exclusively of Watson-Crick base pairs. In the broadest aspect, a complementary sequence is a sequence that forms a duplex without mismatches.

The term complementary sequence has been defined above. The phrase “are capable of base pairing to” is related to the term complementary sequence. I.e. a first sequence is capable of base pairing to a second sequence, which is complementary to the first sequence.

A contiguous stretch of bases is intended to mean a non-interrupted sequence of bases that all fit into a duplex formed between the oligonucleotide and the target RNA. I.e. there are preferably no bulges in the duplex and it is preferred that the sequences are complementary (see the definition of complementary sequences above). Most preferred is perfect Watson-Crick duplex between the oligonucleotide of the invention and target region of the target RNA.

The terms contiguous and continuous are used interchangeably herein.

### SUMMARY OF THE INVENTION

The present invention provides bivalent molecules comprising a first oligonucleotide linked to a second oligonucleotide.

The first and the second oligonucleotide are preferably linked via a linking moiety.

Preferably, both the first and/or the second oligonucleotide comprise an antisense sequence complementary to a cellular RNA such as mRNA or microRNA.

The antisense sequence may be a Blockmir antisense sequence capable of binding to a microRNA binding site in a target RNA. The antisense sequence may also be an antimir antisense sequence capable of binding to a microRNA. It is preferred that the first oligonucleotide and/or the second oligonucleotide comprise a seed sequence of microRNA, a sequence capable of base pairing to the complementary sequence of a seed sequence or a sequence capable of base pairing to a seed sequence.

The bivalent molecules of the invention are useful for modulating microRNA regulatory pathways and may be used e.g. in research and as therapeutics.

They may be used to block microRNA activity by binding to microRNA to thereby deregulate all targets of the microRNA. Importantly, the bivalent molecules of the invention may bind to (and inhibit or inactivate) two identical microRNAs (or microRNA families) or to two different microRNAs (or microRNA families).

They may also bind to microRNA binding site(s) in a target RNA to thereby prevent microRNA binding to the given microRNA binding site. This will prevent microRNA regulation of only the blocked target RNA, while other target RNAs of the microRNA can be left unaffected by the bivalent molecule.

In yet another embodiment, the first oligonucleotide of the molecule may bind a microRNA and the other oligonucleotide of the molecule may bind a microRNA binding site. In this way, a microRNA may be tethered to a mRNA via the bivalent molecule to impose microRNA regulation of the given mRNA.

### DETAILED DESCRIPTION

Bivalent Molecule

A first aspect of the present invention is a bivalent molecule comprising a first oligonucleotide linked to a second oligonucleotide.

Preferably, the first oligonucleotide and/or the second oligonucleotide is not any of or is not selected from the group consisting of an aptamer, siRNA, ribozyme, RNase H activating antisense oligonucleotide, full unmodified RNA oligonucleotide or full unmodified DNA oligonucleotide and it is preferred that the antisense oligonucleotides of the molecules of the invention are preferably not capable of recruiting RNase H and/or RISC (the RNAi machinery).

Instead, it is preferred that the first and/or the second oligonucleotide comprise an antisense sequence complementary to a cellular RNA such as mRNA or microRNA and that the antisense sequences act as simple steric blockers.

The antisense sequence may be a Blockmir antisense sequence capable of binding to a microRNA binding site in a target RNA. The antisense sequence may also be an antimir antisense sequence capable of binding to a microRNA.

It is preferred that the first oligonucleotide and/or second oligonucleotide comprise a seed sequence (or a part of a seed sequence) of a microRNA, a sequence capable of base pairing to the complementary sequence of a seed sequence or a sequence capable of base pairing to a seed sequence.

Preferred microRNAs are human microRNAs and preferred mRNAs are also human.

Sequences Defined by Complementarity

In a preferred embodiment, the first oligonucleotide and/or the second oligonucleotide of the molecule of the invention comprise
a. A contiguous sequence of at least 5 nucleotides that is capable of base pairing to the complementary sequence of one of SEQ ID NOs 1-723 (Blockmir antisense sequence) or
b. A contiguous sequence of at least 5 nucleotides that is capable of base pairing to one of SEQ ID NOs 1-723 (antimir antisense sequence)
Wherein 1, 2, or 3 A's in any of SEQ ID NOs 1-723 may be substituted with I (inosine) and wherein I base pairs to A, C and U and wherein wobble G-U base pairs are allowed, alternatively
Wherein 1, 2, or 3 A in the SEQ ID NO may not be substituted with I and wherein wobble G-U base pairs are allowed, alternatively
wherein 1, 2, or 3 A in the SEQ ID NO may not be substituted with I and wherein wobble G-U base pairs are not allowed.

As will be recognized, “a contiguous sequence of at least 5 nucleotides that is capable of base pairing to the complementary sequence of one of SEQ ID NOs:1-723” is a sequence that may bind to the same sequence as a microRNA (represented by a given SEQ ID NO). Such sequences may herein be referred to as Blockmir antisense sequences or just Blockmir sequences.

As will be recognized, “A contiguous sequence of at least 5 nucleotides that is capable of base pairing one of SEQ ID NOs 1-723” is a sequence that may bind to a microRNA (represented by a given SEQ ID NO). Such sequences may herein be referred to as antimir antisense sequences or just antimir sequences.

Other preferred contiguous sequences (antimir or Blockmir as described above) is at least 6 nucleotides, at least 7, at least 8, at least 9, at least 10, at least 11, at least 12, at least 13, at least, 14, at least 15, at least 16, at least 17, at least 18, at least 19, at least 20 at least 21, at least 22 nucleotides, no more than 22, no more than 21, no more than 20, no more than 19, no more than 18, no more than 17, no more than 16, no more than 15, no more than 14, no more than 13, no more than 12, no more than 11, no more than 10, no more than 9, no more than 8 nucleotides.

Particular preferred are contiguous sequences (antimir or Blockmir as described above) of between 6 and 18 nucleotides, 7 and 15 nucleotides, 7 and 12 nucleotides, 8 and 12 nucleotides, 7 and 10 nucleotides and 8 and 10 nucleotides.

As mentioned, in one embodiment, both the first and the second oligonucleotide comprise a Blockmir antisense sequence. In another embodiment, both the first and the second oligonucleotide comprise an antimir antisense sequence. And in yet another embodiment, the first oligonucleotide comprises a Blockmir antisense oligonucleotide and the second oligonucleotide comprises an antimir antisense oligonucleotide.

In one embodiment, the first oligonucleotide and/or the second oligonucleotide comprise, or more preferably consist of
a. (blockmir) a sequence selected from the group consisting of contiguous sequences that are capable of base pairing to the complementary sequence of a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723 or
b. (antimir) a sequence selected from the group consisting of contiguous sequences that are capable of base pairing to a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723
Wherein 1, 2, or 3 A's in any of SEQ ID NOs 1-723 may be substituted with I (inosine) and wherein I base pairs to A, C and U and wherein wobble G-U base pairs are allowed, alternatively
Wherein 1, 2, or 3 A in the SEQ ID NO may not be substituted with I and wherein wobble G-U base pairs are allowed, alternatively
wherein 1, 2, or 3 A in the SEQ ID NO may not be substituted with I and wherein wobble G-U base pairs are not allowed.

Alternative Way of Describing Sequences

The antisense sequences of the molecules of the invention can also be described as follows:

Blockmir antisense sequence:

In another preferred embodiment, Blockmir antisense sequences of the molecules of the invention comprises, or more preferably consist of, a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723, wherein
a. A may be exchanged with only G, C, U, T or I
b. G may be exchanged with only A or I
c. C may be exchanged with only A, U or T
d. U may be exchanged with only C, A, T or I
and 3 additional positions may be exchanged with any base.

The exchange rules are based on the following considerations:

An A in the microRNA can base pair to U or I in the target RNA. U and I in the target RNA can base pair to A, G, I, C, U or T. Likewise for the other bases.

Moreover, editing of A to I in microRNAs has been shown to redirect silencing targets of microRNAs (Kawahara Y, 2007). Therefore, A in the microRNAs may be substituted for I some embodiments.

Also the target RNA may comprise I that have been edited from A.

Moreover, G:U base pairs may be accepted for microRNAs—target RNA interaction in some embodiments, but not all.

The rules are described in table 1:

Inosines in target RNA and miRNA + GU basepairs

| | | | | | | |
|---|---|---|---|---|---|---|
| MicroRNA | U | G | C | A | I | A/I |
| target | A, G, I | U, C | G, I | U, I | A, C, U | |
| RNA | | | | | | |
| Xmir | U, I , A, | A, G, I | U, C, | A, G, I, | U, I, A, | A, G, |
| | C, T | | A, T | C, U, T | G, T | I, C, |
| | | | | | | U, T |

Inosines in target RNA and miRNA + GU pairs, no T-I pairs

| | | | | | | |
|---|---|---|---|---|---|---|
| MicroRNA | U | G | C | A | I | A/I |
| target | A, G, I | U, C | G, I | U, I | A, C, U | |
| RNA | | | | | | |
| Xmir | U, I, A, | A, G, I | U, C, | A, G, I, | U, I, A, | A, G, |
| | C, T | | A | C, U | G, T | I, C, |
| | | | | | | U, T |

Inosines in target RNA and miRNA, no GU basepairs

| | | | | | | |
|---|---|---|---|---|---|---|
| MicroRNA | U | G | C | A | I | A/I |
| target | A, I | C | G, I | U, I | A, C, U | |
| RNA | | | | | | |
| Xmir | U, I, A, | G, I | A, C, | A, I, C, | U, I, G, | A, G, |
| | C, T | | U, T | U, T | A, T | I, C, |
| | | | | | | U, T |

Inosines in target RNA and miRNA, no GU pairs, no I-T pairs

| | | | | | | |
|---|---|---|---|---|---|---|
| MicroRNA | U | G | C | A | I | A/I |
| target | A, I | C | G, I | U, I | A, C, U | |
| RNA | | | | | | |
| Xmir | U, I, A, | G, I | A, C, U | A, I, | U, I, G, | A, G, |
| | C, T | | | C, U | A, T | I, C, |
| | | | | | | U, T |

No inosine in target RNA

| | | | | | | |
|---|---|---|---|---|---|---|
| MicroRNA | U | G | C | A | I | A/I |
| target | A, G | U, C | G, I | U | A, C, U | |
| RNA | | | | | | |
| Xmir | U, C, T | A, G, I | U, C, | A, G, I | U, G, I, | U, G, |
| | | | A, T | | A, T | I, A, |
| | | | | | | T |

No inosine in either target RNA or miRNA

| | | | | |
|---|---|---|---|---|
| MicroRNA | U | G | C | A |
| target | A, G | U, C | G | U |
| RNA | | | | |
| Xmir | U, C, T | A, G | U, C, T | A, G |

No GU pairs and no inosine in either target RNA or miRNA

| | | | | |
|---|---|---|---|---|
| MicroRNA | U | G | C | A |
| target | A | C | G | U |
| RNA | | | | |
| Xmir | U, T | G | C | A |

Additional positions that may be exchanged with any base are included to account for single nucleotide polymorphisms (SNPs) and other mutations. Furthermore, some target sequences interacting with microRNAs may not posses' perfect complementarity to the interacting microRNA. I.e. there may be a mismatch in the complex formed between the seed sequence of the microRNA and the antiseed sequence of the target RNA.

Thus, in another preferred embodiment,
a. A may be exchanged with only G, C, U, T or I
b. G may be exchanged with only A or I
c. C may be exchanged with only A or U
d. U may be exchanged with only C, A, T or I
and 3 additional positions may be exchanged with any base.

In yet another preferred embodiment,
a. A may be exchanged with only C, U, T or I
b. G may be exchanged with only I
c. C may be exchanged with only A, U or T
d. U may be exchanged with only C, A, T or I
and 3 additional positions may be exchanged with any base.

In yet another preferred embodiment,
a. A may be exchanged with only C, U, or I
b. G may be exchanged with only I
c. C may be exchanged with only A or U
d. U may be exchanged with only C, A, T or I
and 3 additional positions may be exchanged with any base.

In yet another preferred embodiment,
a. A may be exchanged with only G or I
b. G may be exchanged with only I or A
c. C may be exchanged with only A, U or T
d. U may be exchanged with only C or T
and 3 additional positions may be exchanged with any base.

In yet another preferred embodiment,
a. A may be exchanged with only G
b. G may be exchanged with only A or G
c. C may be exchanged with only T or U
d. U may be exchanged with only C or T
and 3 additional positions may be exchanged with any base.

In yet another preferred embodiment, U may be exchanged with only T
and 3 additional positions may be exchanged with any base.

In yet another preferred embodiment, 2 additional positions may be exchanged with any base.

In yet another preferred embodiment, 1 additional position may be exchanged with any base.

In yet another preferred embodiment, no additional positions may be exchanged with any base.

In a preferred embodiment, the first and/or second oligonucleotides may further comprise 1 or 2 additions or deletions. More preferred is 1 addition/substitution and most preferred is zero additions/deletions. Additions and deletions are relevant where the complex between the microRNA and target RNA comprise bulges. If a nucleotide on the microRNA is bulged, this accounts to a deletion of the blockmir antisense sequence of the molecules of the invention. If a nucleotide on the target RNA is bulged, this accounts for an addition of the oligonucleotide of the blockmir antisense sequence of the molecules of the invention.

Antimir antisense sequence:

A in the microRNA may be edited to I, therefore an antimir may have A, C or U in the position corresponding to an A in a microRNA.

Thus, in another preferred embodiment, antimir antisense sequences of the molecules of the invention comprises, or more preferably consist of, a sequence selected from the group consisting of sequences capable of basepairing to position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723, wherein 1, 2 or 3 A's may be substituted with I.

Particular preferred positions are described below.

More Preferred Sequences

The seed sequence of microRNAs is particular important for microRNA binding (and regulation) to its target RNAs.

Therefore, it is particular preferred that Blockmir antisense sequences comprise a sequence selected from the group consisting of contiguous sequences that are capable of base pairing to the complementary sequence of a sequence selected from the group consisting of: position 1-10, position 1-9, position 1-8, position 1-7, position 1-6, position 2-10, position 2-9, position 2-8, position 2-7, position 2-6, position 3-10 and position 3-9 of any SEQ ID NOs:1-723
Wherein 1, 2, or 3 A's in any of SEQ ID NOs 1-723 may be substituted with I and wherein I base pairs to A, C and U and wherein wobble G-U base pairs are allowed, alternatively
Wherein 1, 2, or 3 A in the SEQ ID NO may not be substituted with I and wherein wobble G-U base pairs are allowed, alternatively
wherein 1, 2, or 3 A in the SEQ ID NO may not be substituted with I and wherein wobble G-U base pairs are not allowed alternatively

Alternatively, it is to be understood that the exchange rules outlined above (under alternative way of describing sequences) may be applied for this group, i.e. in its various embodiments.

Most preferred are position 1-8, position 1-7, position 2-9, position 2-8 and position 2-7.

Likewise, it is preferred that antimir antisense sequences comprise a sequence that is capable of base pairing to a sequence selected from the group consisting of: position 1-10, position 1-9, position 1-8, position 1-7, position 1-6, position 2-10, position 2-9, position 2-8, position 2-7, position 2-6, position 3-10 and position 3-9 of any SEQ ID NOs:1-723.

Most preferred is position 1-8, position 1-7, position 2-9, position 2-8 and position 2-7.

In one embodiment, the Blockmir antisense sequence does not comprise the neighbouring nucleotide of either side of the aforementioned positions of any of SEQ ID NOs 1-723. I.e. the neighbouring positions of any of the aforementioned positions of any of SEQ ID NOs 1-723 (when present in a Blockmir antisense sequence) are not the same as the corresponding neighbouring positions of SEQ ID NOs 1-723. In another embodiment, the two neighbouring nucleotide positions of any of the aforementioned positions of any of SEQ ID NOs 1-723 (when present in a Blockmir antisense sequence) are not the same as the corresponding positions in SEQ ID NOs 1-723. This feature is based on the consideration that microRNAs typically do not have perfect complementary to their binding sites in target RNAs, but often do have one with region with perfect complementarity (most often the seed sequence) and modest complementarity for the rest of the microRNA.

Preferably, the Blockmir antisense oligonucleotide can interact with the same region of the target RNA as a microRNA. One advantage of such an oligonucleotide is that it targets an exposed region of the target RNA. Another advantage of such an oligonucleotide is that is can be used to mask the microRNA target such that the (endogenous) microRNA targeting the target RNA will be prevented from interacting with the target RNA, and thus exerts its effects on the target RNA. Importantly, this particular microRNA can be prevented from exerting its effects on this particular target RNA (or particular microRNA binding site if there are more than one binding site for the same microRNA in the same target RNA), while being unaffected in terms of its regulation of its other target RNAs.

As is well known, antimir sequences bind to microRNAs to prevent the microRNA from binding to all its targets.

The oligonucleotides, Blockmir or antimir, of the molecules of the invention may have a degree of identity to any of SEQ ID NOs 1-723 or a complementary thereof selected from the group consisting of less than 99%, less than 95%, less than 90%, less than 85%, less than 80%, less than 75%, less than 70%, less than 65%, less than 60%, less than 55%, less than 50%, less than 45%, less than 40%, less than 35%, less than 30% and less than 25%. When referring to the degree of identity, the degree is counted over the length of the shortest of the SEQ ID NO and the oligonucleotides of the molecules of the invention. Hence, if the SEQ ID NO is 20 bases and the oligonucleotide is 14 and the number of identical positions are 12, the degree of identity is 12/14=86%. If the SEQ ID NO is 20, the oligonucleotide 20 and the number of identical positions is 10, then the degree of identity is 10/20=50%.

For antimir antisense oligonucleotides, identity is typically 100%.

As mentioned above, identity is typically much less for Blockmir antisense oligonucleotides, although this depends on the length of the Blockmir.

### Lengths of Oligonucleotides

The length of the oligonucleotides of the molecules of the invention may be adjusted for various purposes. A stronger interaction with the target RNA may be achieved by increasing the length of the oligonucleotides. On the other hand, the length may be decreased for better delivery and bioavailability. A reduced length will give a decreased tm value (melting temperature) of the oligonucleotides (in complex with a complementary RNA or DNA molecule). However, increasing the concentration of the oligonucleotides may be used to counteract this. More preferably, affinity increasing nucleotides and affinity increasing modifications are used.

The length of the first and the second oligonucleotide (individually) is preferably less than 30 nucleotides, even more preferably less than 20 nucleotides and most preferably less than 16 nucleotides.

Likewise the length of the first and the second oligonucleotide is preferably more than 5 nucleotides, 6 nucleotides, 7 nucleotides and 8 nucleotides.

Preferred ranges are between 15 nucleotides and 5 nucleotides, between 14 nucleotides and 5 nucleotides, between 13 nucleotides and 5 nucleotides, between 12 nucleotides and 5 nucleotides, between 11 nucleotides and 5 nucleotides, between 10 nucleotides and 5 nucleotides, between 9 nucleotides and 5 nucleotides, between 8 nucleotides and 5 nucleotides, between 7 nucleotides and nucleotides, between 15 nucleotides and 6 nucleotides, between 14 nucleotides and 6 nucleotides, between 13 nucleotides and 6 nucleotides, between 12 nucleotides and 6 nucleotides, between 11 nucleotides and 6 nucleotides, between 10 nucleotides and 6 nucleotides, between 9 nucleotides and 6 nucleotides, between 8 nucleotides and 6 nucleotides, between 7 nucleotides and 6 nucleotides, between 15 nucleotides and 7 nucleotides, between 14 nucleotides and 7 nucleotides, between 13 nucleotides and 7 nucleotides, between 12 nucleotides and 7 nucleotides, between 11 nucleotides and 7 nucleotides, between 10 nucleotides and 7 nucleotides, between 9 nucleotides and 7 nucleotides and between 8 nucleotides and 7 nucleotides.

### Very Short Fully Modified Oligonucleotides

One advantage of the present invention is that it enables the use of very short oligonucleotides, because the first and the second oligonucleotide will bind cooperatively to their target RNAs.

When both the first and the second oligonucleotide binds to the same target RNA (same entity), the binding energy for each oligonucleotide can be added (giving an exponential increase in binding affinity) and hence it may be said that the oligonucleotides will bind cooperatively (the first oligonucleotide significantly increases the binding affinity of the second oligonucleotide and vice versa). It should be recognized though that the term cooperative may be misleading in this context because the first and the second oligonucleotide is part of the same molecule. However, if the first and the second oligonucleotide are regarded as separate entities, it is clear that they will bind cooperatively. Moreover, if the first and the second oligonucleotide are tested individually in terms of binding to a target RNA, they will have much reduced affinity as compared to the bivalent counterpart and most often, they will also have reduced activity.

When the first and the second oligonucleotide binds to two separate microRNAs (identical or different), cooperativity is expected because microRNAs in general bind cooperatively to target RNAs. I.e. a first microRNA bound to a given target RNA typically facilitates binding of a second microRNA to the same target RNA. Not intended to be bound by theory, it is believed that a first and second microRNA bound the same target RNA often interacts to create additional binding energy and hence cooperative binding.

Thus, if the first and the second oligonucleotide are tested individually in terms of binding to a target RNA, they will have much reduced affinity as compared to the bivalent counterpart and most often, they will also have reduced activity if they have any activity at all.

In addition to the advantages regarding binding affinities, the molecules of the invention also have other specific advantages e.g. relating to biodistribution in the organism as well as within organs and single cells. This particular applies for the use of a very short first and/or second oligonucleotide. Moreover, advantages in terms of duration of action may be observed, possibly caused by improved biostability.

If the oligonucleotides are 15 or shorter, they may be fully modified with affinity increasing nucleotide analogues (e.g. LNA or other 2′-O-modifications). This becomes increasingly relevant with decreasing length.

Thus, in a preferred embodiment, the bivalent molecules of the invention may comprise a first oligonucleotide of e.g. 8 nucleotides (e.g. LNA or other 2′-O-modified nucleotides) complementary to position 2-9 of a first microRNA and a second oligonucleotide of e.g. 8 nucleotides (e.g. LNA or other 2′-O-modified nucleotides) complementary to position 2-9 of a second microRNA. The first and the second microRNA may be the same or they may be different. Importantly, when using very short antisense sequences, microRNA families sharing the same seed sequence may be targeted. I.e. the molecules of the invention enable targeting of two different microRNAs or two different microRNA families with the same molecule. This cannot be achieved using the molecules currently part of the state of the art, in particular not exogenously synthesized molecules comprising less than 30 or 20 nucleotides.

In another preferred embodiment, the first oligonucleotide may consist of a Blockmir antisense sequence of a length of 7-9 nucleotides (e.g. LNA or other 2′-O-modified nucleotides, specific sequences are given above) comprising the seed sequence of a first microRNA and second oligonucleotide may consist of a Blockmir antisense sequence of a length of 7-9 nucleotides (e.g. LNA or other 2′-O-modified nucleotides) comprising the seed sequence of a second microRNA, wherein the first and the second microRNA may be different or identical. Thus, if a given microRNA has two binding sites in the same target RNA, both binding sites may both be blocked using the same bivalent molecule. Likewise if the same target RNA is regulated by two different microRNAs, both microRNA binding sites may be blocked by the same bivalent molecule. This cannot be achieved using the molecules currently part of the state of the art, in particular not exogenously synthesized molecules comprising less than 30 or 20 nucleotides

### Activity of Oligonucleotides

As mentioned above, it is preferred that the first and the second oligonucleotide do not recruit the RNAi machinery or RNase H. Likewise, the oligonucleotides should not act as a ribozyme, DNAzyme or aptamer.

Instead, it is preferred that the oligonucleotides are steric blockers. This can be achieved by a modification pattern that makes the oligonucleotide incompatible with RNase H and the RNAi machinery as is further described below.

### RNase H Cleavage

RNase H cleaves the RNA part of a RNA-DNA duplex. The structural requirements for RNase H activation are well-known to the skilled man. This mechanism is very often used to achieve traditional antisense regulation e.g. by employing so-called gapmers. Gapmers are antisense oligonucleotides that comprise a central region with deoxy sugars (the gap) and modified flanks. Gapmers very often comprises phosphorothioate internucleotide linkages to improve biostability and the flanks comprise e.g. 2-O-modifications that also improve biostability, i.e. resistance against nucleolytic attack and increase the melting temperature of the gapmer base paired to a complementary nucleic acid. Also headmer and endmer structures have been described in the literature.

As mentioned it is preferred that the oligonucleotide of the molecules of the invention is not capable of inducing RNase H cleavage of the target RNA. The skilled man is well aware of the requirements for RNase H cleavage and will be able to design oligonucleotides that do or do not activate RNase H. The skilled man will also be capable of testing whether oligonucleotides do or do not activate RNase H. Most importantly, the oligonucleotide should not contain extended stretches of unmodified DNA.

Thus, it is preferred that the oligonucleotide does not comprise a stretch of unmodified DNA that exceeds a length selected from the group consisting of: 3 bases, 4 bases, 5 bases, 6 bases, 7 bases, 8 bases, 9 bases, 10 bases and 11 bases. Most preferably, the stretch of unmodified DNA does not exceed 3 bases. In another preferred embodiment, the oligonucleotide does not comprise any DNA monomers.

A positive description of all allowed modifications that will prevent RNase H activation is not feasible, since a very wide variety of modifications will do that. Particular preferred modifications and patterns are described below.

### RNAi Machinery

The RNAi machinery is a sophisticated gene regulatory system that is guided by RNA. Thus, microRNAs guide the RNAi machinery to target mRNAs to affect the activity of the target mRNA. The RNAi machinery may affect translation of the mRNA directly or it may affect the stability of the target mRNA, i.e. mediate direct degradation of the target mRNA. Not intended to be bound by theory, it is believed that the degree of complementarity between microRNA and target mRNA is a key element as to whether the target mRNA is subjected to translational regulation or degradation.

Endogenous microRNAs are processed from precursor stem-loops and incorporated into a so called RNA induced silencing complex (RISC complex). The details of this process are still poorly understood.

The cellular RNAi machinery has been extensively used to affect the activity of cellular mRNAs by introducing synthetic double stranded RNA complexes termed siRNAs into the cell. As mentioned above, siRNAs are short double stranded RNA complexes comprising a passenger strand and a complementary guide strand. The guide strand of siRNA is incorporated into the RISC complex, where after the RISC complex can affect the activity of mRNA harbouring complementary sequences to the guide strand. Thus, siRNAs are a new class of compounds that is thought to be capable of efficiently and specifically targeting any mRNA and consequently, siRNAs are regarded potentially as a new class of therapeutics.

A common feature of siRNAs and microRNAs is that they recruit the cellular RNAi machinery to affect the activity of target RNAs.

As mentioned, it is preferred that the oligonucleotides of the molecules of the invention are not capable of recruiting the RNAi machinery and hence direct the RNAi machinery to the target RNA. I.e. the oligonucleotides of the molecules of the invention should not be designed as siRNA or microRNA.

The skilled man is well aware of the requirements for recruitment of the RNAi machinery and will be able to design oligonucleotides that do or do not recruit the RNAi machinery. Moreover, the skilled man will be capable of testing whether oligonucleotides do or do not recruit the RNAi machinery.

It is particular preferred that the oligonucleotides are single stranded and that they are not fully RNA—as opposed to a siRNA designed for recruiting the RNAi machinery.

In one embodiment the oligonucleotide does not comprise a stretch of unmodified RNA monomers that exceeds a length selected from the group consisting of: 3 bases, 4 bases, 5 bases, 6 bases, 7 bases, 8 bases, 9 bases, 10 bases and 11 bases. Most preferably, the stretch of unmodified RNA does not exceed 3 bases. This will ensure that the oligonucleotide does not recruit the RNAi machinery.

However, it must be recognized that in some embodiments, the oligonucleotide can indeed comprise more than 3 contiguous RNA units. In such embodiment, heavy modification of the rest of the oligonucleotide may be used to prevent recruitment of the RNAi machinery.

In another preferred embodiment, the oligonucleotide does not comprise any RNA monomers.

A positive description of all allowed modifications that will prevent recruitment of the RNAi machinery is not feasible, since a very wide variety of modifications will do that. Particular preferred modifications and patterns are described below.

### Chemistry and Architecture

As described above, it is preferred that the oligonucleotides of the molecules of the invention do not recruit the RNAi machinery and at the same time do not recruit RNase H. Moreover, it is desired that the oligonucleotides have a sufficient bioavailability and stability. These characteristics can be achieved by appropriate chemical modifications.

Since only very specific oligonucleotide architectures and chemistry allow recruitment of RNase H and the RNAi machinery, the oligonucleotides of the molecules of the invention are best described by way of non-allowed structures or negative limitations as described above.

Hereafter, a number of allowed and preferred modifications are described. Again it is emphasized that it is impossible to exhaustively describe all allowed modifications which will enable the oligonucleotides to act as steric blockers. In general it may be said that this can be achieved by a modification pattern that makes the oligonucleotide incompatible with RNase H and the RNAi machinery.

### Nucleotide Analogues and Modifications

As mentioned, it is preferred that the first and/or second oligonucleotide comprise nucleotide analogues such as to improve affinity, bioavailability and biostability and also to prevent recruitment of the RNAi machinery and RNase H activation.

Preferred nucleotide analogues are e.g. RNA units modified in the 2-O-position (e.g. 2′-O-(2-methoxyethyl)-RNA, 2′O-methyl-RNA, 2′O-flouro-RNA), locked nucleic acid (LNA) units (e.g. thio-, amino- and oxy-LNA and L-ribo-LNA), intercalating nucleic acid (INA) units, morpholino units, PNA (peptide nucleic acid) units, 2′-Deoxy-2′-fluoro-arabinonucleic acid (FANA), arabinonucleic acid (ANA), unlocked nucleic acid (UNA) units and Hexitol nucleic acid (HNA) units.

The first and/or the second oligonucleotide may e.g. comprise 1, 2, 3 or 4 of the above listed nucleotide analogues.

In one embodiment, the first and/or the second oligonucleotide does not comprise 1, 2, 3 or 4 nucleotide analogues selected from the group consisting of RNA units modified in the 2-O-position (e.g. 2′-O-(2-methoxyethyl)-RNA, 2′O-methyl-RNA, 2′O-flouro-RNA), locked nucleic acid (LNA) units (e.g. thio-, amino- and oxy-LNA and L-ribo-LNA), intercalating nucleic acid (INA) units, morpholino units, PNA (peptide nucleic acid) units, 2′-Deoxy-2′-fluoro-arabinonucleic acid (FANA), arabinonucleic acid (ANA), unlocked nucleic acid (UNA) units and Hexitol nucleic acid (HNA) units.

Preferred modifications are those that increase the affinity of the oligonucleotide for complementary sequences, i.e. increases the tm (melting temperature) of the oligonucleotide base paired to a complementary sequence.

Such modifications include 2′-O-Flouro, 2′-O-methyl, 2′-O-methoxyethyl, LNA (locked nucleic acid) units, PNA (peptide nucleic acid) units and INA (intercalating nucleic acid) units.

In one embodiment, the number of nucleotide units in the first and/or second oligonucleotide that increase the affinity for complementary sequences is selected from the group of: 1 units, 2 units, 3 units, 4 units, 5 units, 6 units, 7 units, 8 units, 9 units, 10 units, 11 units, 12 units, 13 units, 14 units, 15 units, 16 units, 17 units, 18 units, 19 units, 20 units, 21 units, and 22 units

Shorter oligonucleotides will typically comprise a higher content of nucleotide analogues such as to still allow the oligonucleotide to bind to a complementary nucleic acid. Thus, if the oligonucleotide is less than 12, 11, 10 or 9 units it may consist entirely of nucleotide analogues that increase binding affinity such as LNA.

In one embodiment, the fraction of units modified at either the base or sugar (e.g. LNA or 2′O-methyl-RNA or as mentioned above) relatively to the units not modified at either the base or sugar is selected from the group consisting of 100%, less than 99%, 95%, less than 90%, less than 85% or less than 75%, less than 70%, less than 65%, less than 60%, less than 50%, less than 45%, less than 40%, less than 35%, less than 30%, less than 25%, less than 20%, less than 15%, less than 10%, and less than 5%, less than 1%, more than 99%, more than 95%, more than 90%, more than 85% or more than 75%, more than 70%, more than 65%, more than 60%, more than 50%, more than 45%, more than 40%, more than 35%, more than 30%, more than 25%, more than 20%, more than 15%, more than 10%, and more than 5% and more than 1%.

Typically the fraction of units modified at either the base or sugar relatively to the units not modified at either the base or sugar will be between 50% and 100% and even more preferred between 75% and 100%.

Further, in a preferred embodiment, phosphorothioate internucleotide linkages may connect the units to improve the biostability of the oligonucleotide. Thus, the first and/or the second oligonucleotide may be fully phosphorothiolated or only partly phosphorothiolated.

In another embodiment, the fraction of phosphorothioate linkages is selected from the group consisting of 100%, less than 95%, less than 90%, less than 85% or less than 75%, less than 70%, less than 65%, less than 60%, less than 50%, more than 95%, more than 90%, more than 85% or more than 75%, more than 70%, more than 65%, more than 60% and more than 50%.

The oligonucleotide may also comprise phosporoamidate linkages, and preferably fraction of phosporoamidate linkages is linkages is selected from the group consisting of 100%, less than 95%, less than 90%, less than 85% or less than 75%, less than 70%, less than 65%, less than 60%, less than 50%, more than 95%, more than 90%, more than 85% or more than 75%, more than 70%, more than 65%, more than 60% and more than 50%.

In yet another embodiment, the first and/or second oligonucleotide comprise less than 8, such as less than 7, less than 6, less than 5 less than 4, less than 3, less than 2 and less than 1 unmodified DNA and/or unmodified RNA units.

As should be clear the modifications and nucleotide analogues may be combined and it should be clear that phosphoroamidate and phosphorothioate modifications can be used in combination with sugar or base modifications at the same unit position. Thus, LNA phosphoromidates may e.g. used.

In a preferred embodiment, the oligonucleotide comprises a repeating pattern of one or more modifications, e.g. LNA units and one or more units that are substituted in the 2′-position. OMe/LNA mixmers have been shown to be powerful reagents for use as steric block inhibitors of gene expression regulated by protein-RNA interactions. Thus, when the oligonucleotides of the invention are used to block the activity of a microRNA at a target RNA, a OMe/LNA mixmer architecture (preferably connected by phosphorothioate linkages) may be used. A gapmer structure may also be used, however preferably without being capable of inducing RNase H if the oligonucleotide is intended to act as a Blockmir.

In another preferred embodiment, the oligonucleotide comprises exclusively LNA units and DNA units and these may be connected by phosphorothioate linkages as outlined above.

In still another embodiment, the first and/or the second oligonucleotide of the invention does not comprise any morpholino units and/or LNA units and/or PNA units and/or 2′-O-modified RNA units and/or unmodified DNA units and/or unmodified RNA units. When reference is made to unmodified DNA and RNA, the interlinkage may (or may not) be e.g. phosphorothioate or phoshoroamidate.

### Linking Moiety

Is it preferred that the first and second oligonucleotide is linked via a linking moiety as opposed the just a covalent bond between the first and the second oligonucleotide, in which case the molecule of the invention is basically the first and the second oligonucleotide being directly linked to form a non-interrupted stretch of nucleotides.

However, in one embodiment, the linker moiety consists of or comprises an oligonucleotide comprising between 1 and 40 contiguous nucleotides, such as between 2 and 15 nucleotides, between 3 and 12 nucleotides, between 4 and 10 nucleotides or between 5 and 8 nucleotides. In this embodiment, the linker may comprise the same kind of nucleotide analogues as the first and/or second oligonucleotide. In a related embodiment the linker may comprise abasic or universal bases. The linker may also exclusively consist of abasic units in which case the linker is just a polymeric sugar phosphate backbone.

It is preferred that the linking moiety is attached to the 3′end of the first oligonucleotide and to the 5′end of the second oligonucleotide.

However, in alternative embodiments the linking moiety may be attached to the 3′end of both the first and the second oligonucleotide, to the 5′end of both the first and the second oligonucleotide. The linking moiety may also be attached to neither the 5′end nucleotide or the 3′end nucleotide, i.e. the linking moiety may be linked internally in the first and/or the second oligonucleotide.

The linking moiety may be attached to the nucleobase or to the sugar phosphate backbone of the first and second oligonucleotide.

The linking moiety may e.g. be a polypeptide, polysaccharide, C8, C6, or C12.

In a preferred embodiment, the linking moiety consist of or comprise a non-nucleotide polymer such as polyalkylen oxide, polyethyleneglcyol for example alpha-, omega-dihydroxypolyethylenglycol, biodegradable lactone-based polymers e.g. polyacrylic acid, polylactide acid (PLA), poly(glycolic acid) (PGA), polypropylene, polystyrene, polyolefin, polyamide, polycyanoacrylate, polyimide, polyethyleneterephtalat (PEY, PETG), polyethylene terephtalate (PETE), polytetramethylene glycol (PTG), polyurethane (as well as mixtures thereof).

Most preferred is polyalkylene glycol such as polyethylene glycol.

Pegylation of oligonucleotides and methods for preparation of pegylated oligonucleotides have e.g. been described in Nucleic Acids Research, 1994, Vol. 22, No. 22, 4810-4817, WO2008/077956 and WO2005/111238 which are all hereby incorporated by reference.

In a preferred embodiment, the linking moiety is attached during oligonucleotide synthesis such that when the first oligonucleotide have been synthesized, the linking moiety is attached to the first oligonucleotide, where after the second oligonucleotide is synthesized. I.e. the linking moiety adapted for use in standard oligonucleotide synthesis may be used.

Examples of commercially available linking moieties adapted for incorporation into an oligonucleotide are:

Spacer 18 amidite (17-O-DMT-Hexaethyleneoxide-1-O-phosphoramidite), Spacer 9 Amidite (8-DMT-O-Triethyleneoxide-1-O-phosphoramidite), C6 Spacer Amidite (6-DMT-O-Hexanediol-1-O-Phosphoramidite) and C3 Spacer Amidite (DMT-1,3 propanediol-phosphoramidite). As will be recognized, multiples of linking moieties may be incorporated to obtain a desired linker length, e.g. between 1 and 100, such as between 1 and 50, between 1 and 25, between 1 and 10, between 1 and 9, between 1 and 8, between 1 and 7, between 1 and 6, between 1 and 5, between 1 and 4, between 1 and 3, and between 1 and 2.

The length of the linking moiety may be adjusted according the specific use of the particular bivalent molecule. If the first and the second oligonucleotide of the molecule comprise Blockmir antisense sequences, the length of the linking moiety may be adjusted according to the distance between the two microRNA binding sites in the target RNA. Thus, if the binding sites are separated by 20 nucleotides, then the linking moiety may have a length between 10 and 70 angstrom based on the distance between nucleotides in a linear nucleic acid. Normally, there should be no reason to use a linker that is significantly longer than the linear distance between binding sites. On the other hand, it should be recognized that the sites may be much closer than the linear distance because of the three dimensional structure of the target RNA. Therefore, it is typically recommended to test various linking moieties with varying lengths. It is generally preferred that the linking moiety is no more than 1000 angstrom in length, such as no more than 900, 800, 700, 600, 500, 400, 300, 200 or 100 angstrom in length. It is also preferred that the linking moiety is at least 10 angstrom in length, such as 15, 20, 25, 30, 35 or angstrom in length.

Preferred ranges are between 10 and 1000 angstrom, between 20 and 500 angstrom and between 20 and 200 angstrom.

When the first and the second oligonucleotide both comprise antimir antisense sequences, the linking moiety will typically have a length between 10 and 100 angstrom, more preferably between 20 and 75 angstrom.

### Delivery

Various methods for delivery of oligonucleotides are known to the skilled man. Thus, oligonucleotides may be formulated in microparticles and nanoparticles. Liposomes are frequently used as delivery vehicle and a variety of liposome delivery systems exist. They may e.g. comprise cationic lipids or neutral lipids. Their size may be varied for various purposes and other components may be included in the liposomes or on the surface of the liposomes. Chitosan nanoparticles have been used for delivery of plasmids and siRNAs to various cells, among them primary cells. Thus, chitosan nanoparticles may also be used for delivery of the oligonucleotides of the invention. Others polymers for delivery are polyethyleneimine (PEI), cyclodextrin, atelocollagen, polyamidoamine (PAMAM) and poly(lactic-co-glycolic acid) (PLGA). Further, oligonucleotides of the invention may be conjugated to cationic peptides that have been shown to facilitate transport into cells. The oligonucleotides may also be conjugated to lipids to facilitate delivery. In particular, cholesterol conjugation has been used to improve antimir delivery.

A second aspect of the invention is the use of the molecule of the invention for modulating microRNA regulation either by blocking a microRNA or by blocking a microRNA binding site in a target RNA, either in vivo or in vitro.

A third aspect of the invention is the molecule of the invention for use in therapy, e.g. treatment of HCV infection.

### EXAMPLES

### Example 1

Bivalent molecules for treatment of HCV.

### Blockmirs:

### Background

It has been demonstrated that mir-122 modulates Hepatitis C virus RNA abundance by facilitating replication of the viral RNA (Jopling C L, 2005).

The 5′UTR of the HCV genom comprises two conserved antiseed sequence capable of base pairing with the seed sequence of microRNA-122.

It has been demonstrated that the level of HCV viral replicon RNA was reduced by app. 80% when mir-122 was inactivated by a so-called antagomir.

A genetic interaction between mir-122 and the 5′ noncoding region of the viral genom was revealed by mutational analysis of the predicted micro RNA binding site and ectopic expression of mir-122 molecules containing compensatory mutations.

Bivalent Antimirs Targeting microRNA-122

A described, antimir inactivation of microRNA-122 has been demonstrated and microRNA-122 inactivation affects HCV replication. As an alternative, bivalent molecules comprising a first and/or a second antisense sequence directed to microRNA-122 may be employed. Such bivalent molecules may be more potent that than monovalent antimirs because two microRNAs will bind cooperatively to the bivalent molecule. Moreover, they may also have a more favourable biodistribution, because the bivalent molecules in some aspects may have the characteristics of the overall size of the molecule, while in other aspects, the bivalent molecules may have characteristics of the smaller (first and second) oligonucleotides of the bivalent molecule. This may e.g. be the case with respect to entry into cells.

The sequence of mir-122 with the seed sequence underlined is:

3′ UGUUUGUGGUAACAGUGUGAGGU

Base paired to a complementary sequence:

3′ UGUUUGUGGUAACAGUGUGAGGU
5′ ACAAACACCATTGTCACACTCCA

Three bivalent molecules targeting microRNA-122 may e.g. be:

| | |
|---|---|
| a) | TCACACTCC-----TCACACTCC |
| b) | CACACTCC-----CACACTCC |
| c) | TCACACTCC-----CACACTCC |

Wherein ----- denote a linker, e.g. a PEG linker.

The oligonucleotides may e.g. consist entirely of LNA monomers.

More specific embodiments are described in the detailed description.

### Bivalent Blockmirs Targeting HCV

The sequence of the target region (anti-seed sequence is bold) in the 5′noncoding region is:

CCAGCCCCCTGATGGGGGCGACACTCCACCATGAATCACTCCCCTGTGAG
GAACTACTGT

And with the complementary sequence indicated:

5′ CCAGCCCCCTGATGGGGGCGACACTCCACCATGAATCACTCCCCTGT
GAGGAACTACT
3′ GGTCGGGGGACTACCCCCGCTGTGAGGTGGTACTTAGTGAGGGGACA
CTCCTTGATGA

Bivalent molecules may e.g. be:

```
5′ CCAGCCCCCTGATGGGGGCGACACTCCACCATGAATCACTCCCCTGTGAGGAACTACT
                                          GCTGTGAGGT-------AGTGAGG5′
5′ CCAGCCCCCTGATGGGGGCGACACTCCACCATGAATCACTCCCCTGTGAGGAACTACT
                                              TGTGAGGT------TAGTGAGG5′
```

Wherein ----- denote a linker, e.g. a PEG linker.

The oligonucleotides may e.g. consist entirely of LNA monomers.

More specific embodiments are described in the detailed description.

### Example 2

### Bivalent Molecules for Treatment of Cancer

MicroRNA-21 plays is upregulated in various cancers and therefore there is interest in down regulation of microRNA-21.

The sequence of microRNA-21 is:

3′ AGUUGUAGUCAGACUAUUCGAU

Basepaired to a complementary sequence:

3′ AGUUGUAGUCAGACUAUUCGAU
5′ TCAACATCAGTCTGATAAGCTA:

Four bivalent molecules targeting microRNA-122 may e.g. be:

| | |
|---|---|
| a) | TGATAAGCT-----TGATAAGCT |
| b) | GATAAGCT-----GATAAGCT |
| c) | ATAAGCT-----ATAAGCT |
| d) | TGATAAGCT-----ATAAGCT |

Wherein ----- denote a linker, e.g. a PEG linker.

The oligonucleotides may e.g. consist entirely of LNA.

More specific embodiments are described in the detailed description.

### Example 3

### Bivalent Molecules Useful for Treatment of Psoriasis.

It has been demonstrated that psoriasis is characterized by a specific miRNA expression profile that differs from that of healthy skin or another chronic inflammatory disease, atopic eczema. Among miRNAs overexpressed in psoriasis, a keratino cytespecific miRNA (miR-203) and a leukocyte-derived miRNA (miR-146a) were identified.

The up-regulation of miR-203 in psoriatic plaques was concurrent with the down-regulation of an evolutionary conserved target of miR-203, suppressor of cytokine signaling 3 (SOCS-3), which is involved in inflammatory responses and keratinocytefunctions (Sonkoly E, 2007, Jul. 11).

Another study showed that miR-146a, one of the psoriasis-specific miRNAs, inhibits the expression of IRAK-1 (interleukin-1 receptor-associated kinase 1) and TRAF-6 (TNF receptor-associated factor 6) proteins both of which are regulators of the TNF-a signalling pathway (Taganov K D, 2006). Hence, it is conceivable that miR-146a is involved in the pathogenesis of psoriasis via the modulation of TNF-a signalling in the skin.

One bivalent molecule can inactivate microRNA-146 and microRNA-203.

The sequences of the microRNAs are:

### Mir-203:

3′ GAUCACCAGGAUUUGUAAAGUG

Base paired to a complementary sequence:

3′ GAUCACCAGGAUUUGUAAAGUG
5′ CTAGTGGTCCTAAACATTTCAC

### Mir-146:

3′ UUGGGUACCUUAAGUCAAGAGU

Base paired to a complementary sequence:

3′ UUGGGUACCUUAAGUCAAGAGU
5′ AACCCATGGAATTCAGTTCTCA

Exemplary bivalent molecules targeting microRNA-203 and microRNA-146a:

| | |
|---|---|
| a) | AACATTTCA-----TCAGTTCTC |
| b) | ACATTTCA-----CAGTTCTC |
| c) | CATTTCA-----AGTTCTC |
| d) | AACATTTCA-----AGTTCTC |
| e) | TCAGTTCTC-----AACATTTCA |
| f) | CAGTTCTC-----ACATTTCA |
| g) | AGTTCTC-----CATTTCA |
| h) | TCAGTTCTC-----CATTTCA |

Wherein ----- denote a linker, e.g. a PEG linker.

The oligonucleotides may e.g. consist entirely of LNA.

More specific embodiments are described in the detailed description.

### Example 4

To test the activity of various bivalent oligonucleotides, a reporter gene construct was made wherein a hepatitis C sequence comprising two microRNA-122 binding sites was cloned behind the renilla luciferase gene in the psiCHECK™-2 Vector. When this plasmid is transfected into cells expressing microRNA-122, or co-transfected with microRNA-122, the microRNA will bind to the binding sites and repress expression of the reporter gene. I.e. the activity of bivalent molecules of the invention targeted to microRNA-122 or the microRNA-122 bindings sites in the reporter construct can easily be tested.

The vector construct was made using the following two oligonucleotides:

HCV-Downstream:

GCCAGCGGCCGGCGGGGAGTGATTCATGGTGGAGTGTCGCCCC

HCV-Upstream:

ATCGCTCGAGGCCAGCCCCCTGATGGGGGCGACACTCCAC

These were annealed and extended in a PCR reaction, where after the double stranded DNA was digested with XhoI and Not-I and cloned into the XhoI and NotI sites of the psiCHECK™-2 Vector.

### Bivalent Oligonucleotides Tested:

(030) ANTIMIR122 CONTROL

5′-LCMCLAMTMTLGLTMCMALCMALCMTLCLC-3′

Antimir control targeting microRNA-122.

(034) HCV Fullmatch 5′-

LGLGMALGMULGMALTMULCMAMULGMGMULGMGLALGMUMGLTLC-3′

Bivalent Blockmir targeted to HCV RNA and which is expected to mask both microRNA-122 binding sites. The linking moiety consists of nucleotides.

(035) All targets full LNA 8-mer 5′-LTLGLGLALGLTLGLT-3′

Monovalent Blockmir with perfect complementarity to target 1 and incomplete complementarity to target 2. See alignment below.

(037) HCV T1 LINK20 T2

5′-LGLGLGLALGLTLGLA3′-X-5′LTLGLGLALGLTLGLT-3′

Bivalent Blockmir targeted to HCV RNA and which is expected to mask both microRNA-122 binding sites. The linking moiety is a PEG linker, see structure below.

(038) HCV T1 LINK40 T2

5′-LGLGLGLALGLTLGLA3′-XX-5′LTLGLGLALGLTLGLT-3′

Bivalent Blockmir targeted to HCV RNA and which is expected to mask both microRNA-122 binding sites. The linking moiety is a PEG linker, see structure below.

(043) Bivalent antimir 21a (linker 20)

5′-LGLALTLALALGLCLT3′-X-5′LGLALTLALALGLCLT-3′

Bivalent antimir targeted to microRNA-21.

(056) MIR122 BIVALENT 20:

5′LCLALCLALCLTLCLC3′-X-5′LCLALCLALCLTLCLC3′

Bivalent antimir targeted to microRNA-122.

(057) MIR122 BIVALENT 40: 5′LCLALCLALCLTLCLC3′-XX-5′LCLALCLALCLTLCLC3′

Bivalent antimir targeted to microRNA-122.

L denote a LNA nucleotide

M denote a 2′O-methyl-RNA nucleotide

X denote a linker:

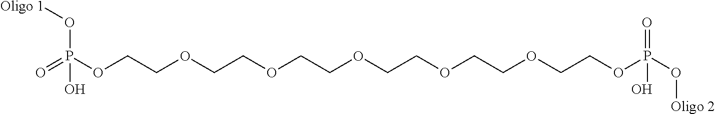

X is incorporated during standard oligonucleotide synthesis using a phosporoamidite:

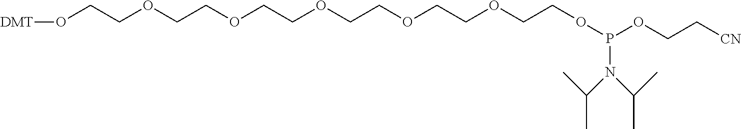

To illustrate where the Blockmirs bind to the HCV targets, the reverse complements of the Blockmirs are here shown aligned with the HCV target sequences.

| | |
|---|---|
| 34 | GACACTCCACCATGAATCACTCC |
| 35 | ACACTCCA ACACTCCA |
| 37 | ACACTCCA---X---ACACTCCC |
| 38 | ACACTCCA---XX--ACACTCCC |
| 1A: | GCCAGCCCCCTGATGGGGGCGACACTCCACCATGAATCACTCCCCTGTGAGGAACTACTGT |
| | binding site 1 binding site 2 |

### Results

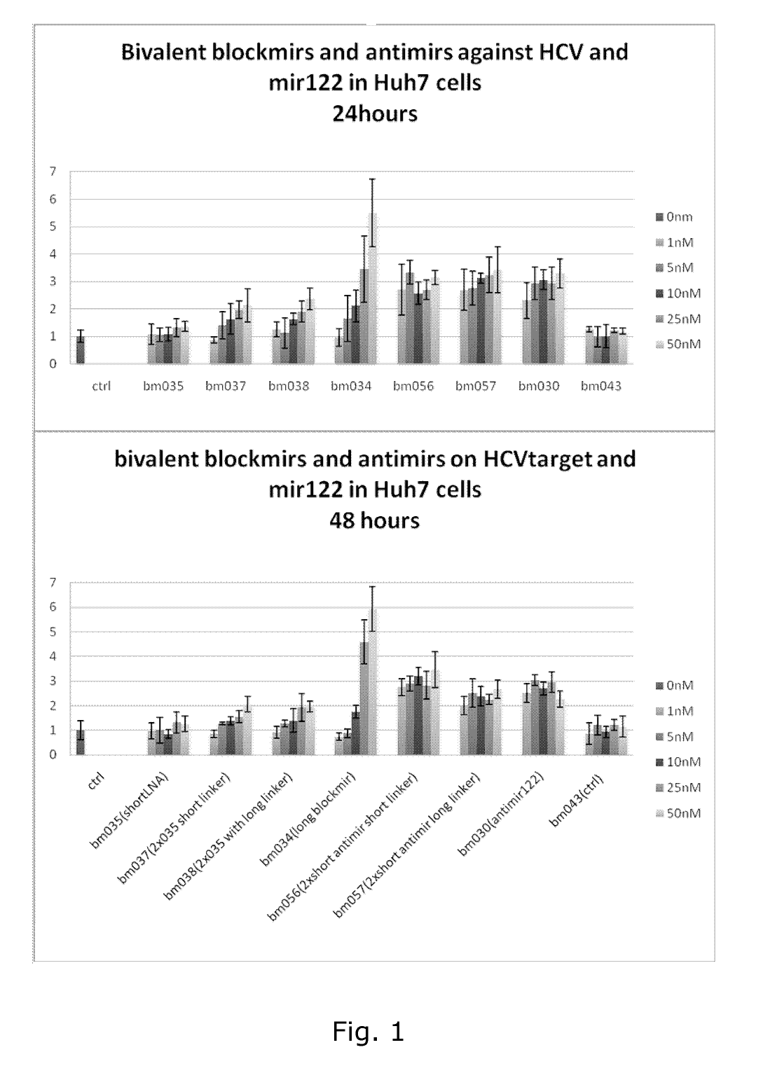

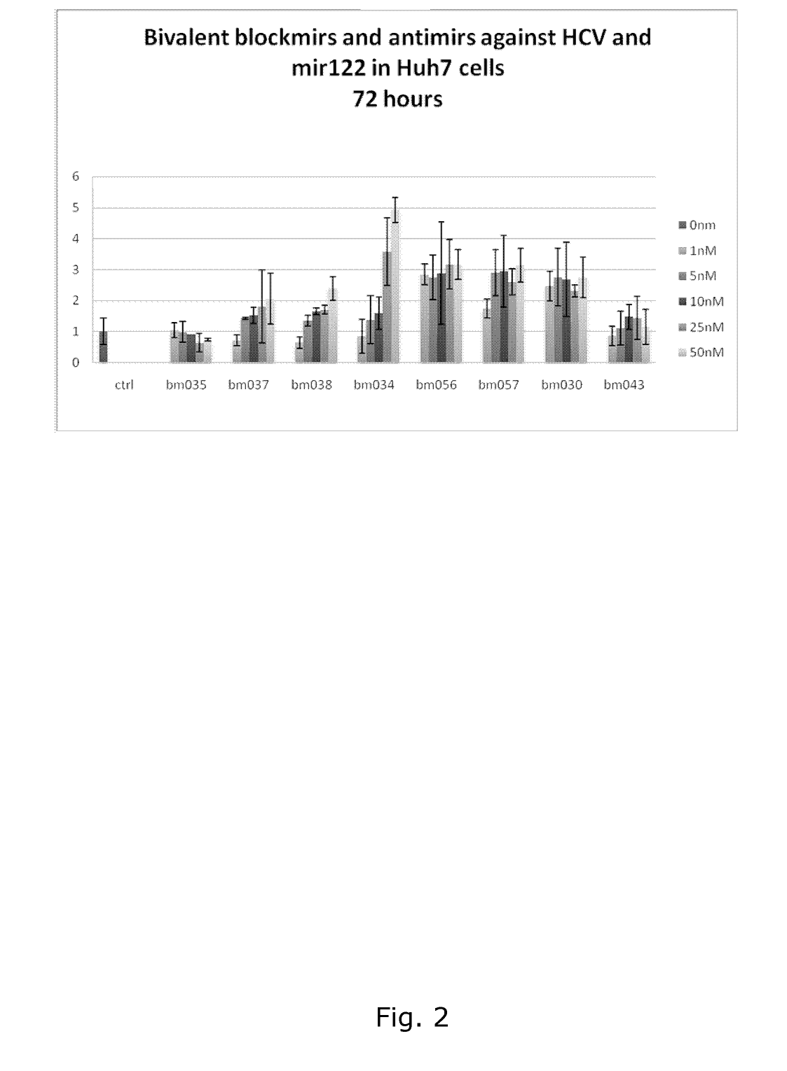

The oligonucleotides and the reporter plasmid was transfected into HUH7 cells expressing microRNA-122 using lipofectamin 2000. Dual luciferase activity (renilla luc vs firefly luc) was measured after 24, 48 and 72 hours. The results are shown in FIGS. 1 and 2. The control bar is mock transfected HUH7 cells, i.e. the control shows the repressed rluc expression. Another control is transfection of 43, which is a bivalent antimir targeted to microRNA-21.

As expected, when the cells have been transfected with a standard antimir (bm030) directed to microRNA-122, rluc is derepressed. When the cells are transfected with bivalent antimirs 56+57, rluc is derepressed with a similar potency as the reference antimir (bm030).

When the cells where transfected with bivalent Blockmirs 37, 38 and 34, in all cases rluc was derepressed, mostly so by Blockmir 34. Importantly, monovalent Blockmir 35 identical to one of the Blockmir antisense oligonucleotides of 34 and had no effect of rluc expression. Thus, going from monovalent to bivalent molecules significantly increased potency.

### Example 5

Since the bivalent antimir molecules of example 4 had approximately the same potency as the reference antimir compound, the activities of the compounds were tested in lower concentrations. In addition, three new bivalent antimir molecules were tested.

### New Bivalent Oligonucleotides Tested:

(120)
LAMCMALCMULCLCMAMCMCMALTMGMAMAMULCMALCMULCLC

Bivalent antimir targeted to microRNA-122. The linking moiety consists of nucleotides. The antiseed sequences are underlined.

(121)
LGMCLCMAMAMCMALCMULCLCMAMCMCMAMUMGMAMAMULCMALCMUMC
LC

Bivalent antimir targeted to microRNA-122. The linking moiety consists of nucleotides. The antiseed sequences are underlined.

(122)
LALCMALCMULCMCMAMCMCMALCMALCMUMCLC

Bivalent antimir targeted to microRNA-122. The linking moiety consists of nucleotides. The antiseed sequences are underlined.

L denote a LNA nucleotide
M denote a 2′O-methyl-RNA nucleotide

### Results

The oligonucleotides were tested as outlined in example 4.

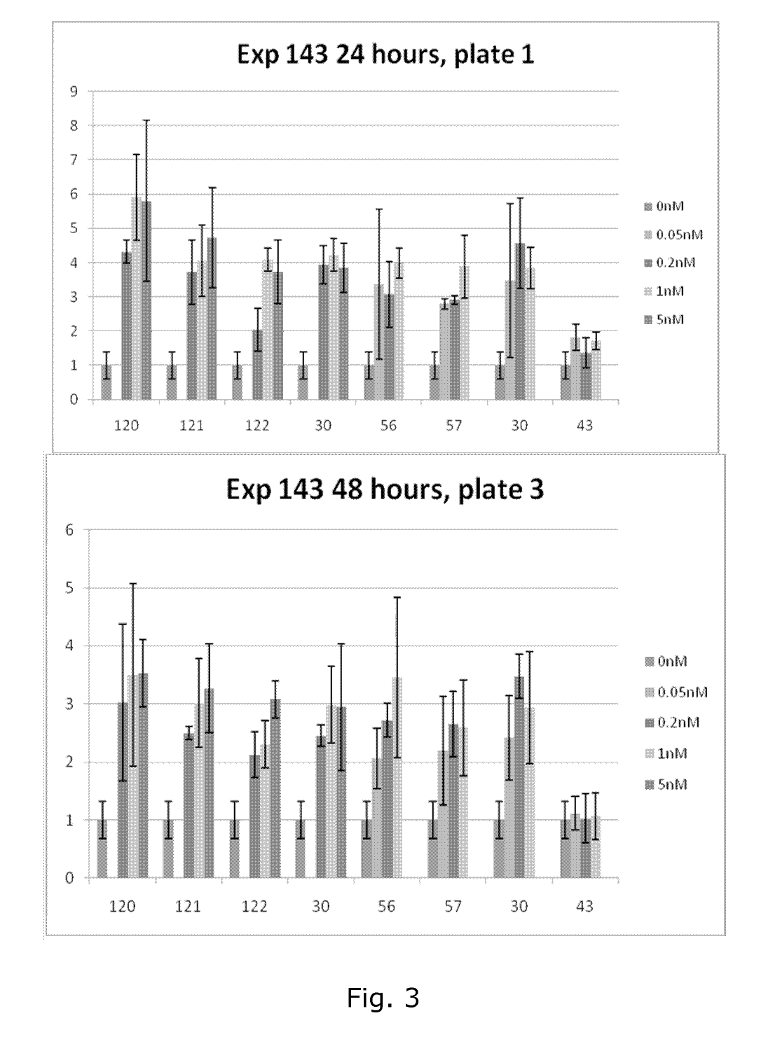

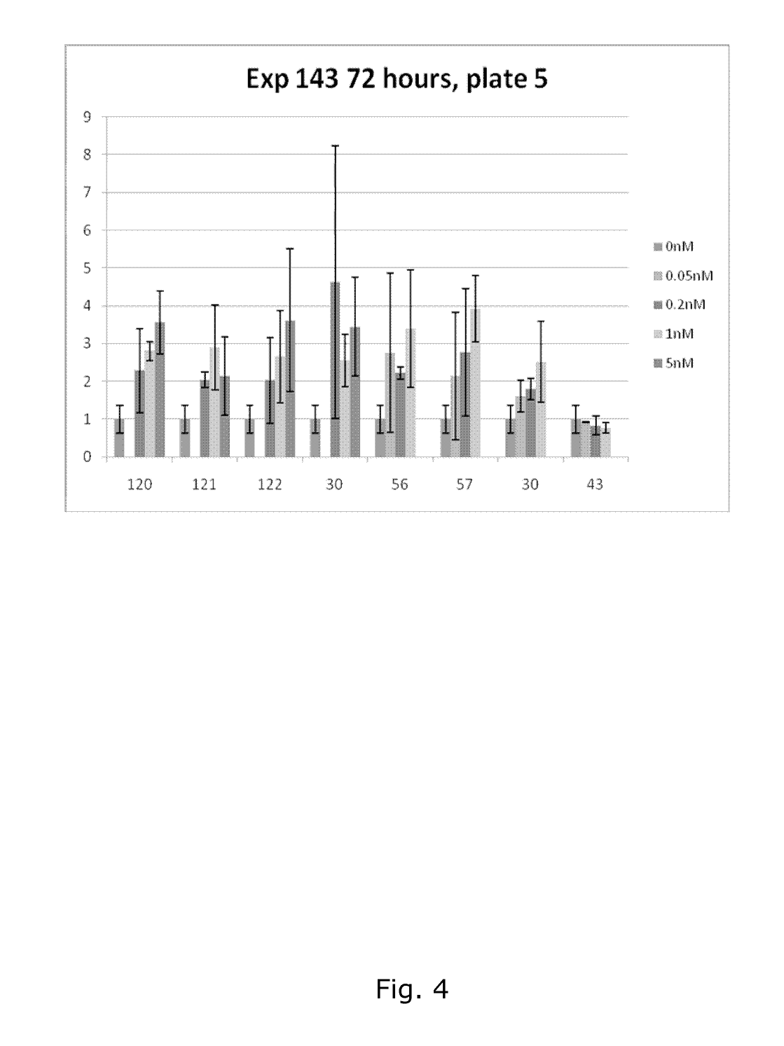

As shown in FIGS. 3 and 4, even at 0.2 nM and 0.05 nM no significant difference in potency was observed between the reference antimir 30 and bivalent antimirs 56 and 57. I.e. the bivalent antimirs were very potent. Moreover, there was a slight tendency for bivalent antimirs 56 and 57 to have a longer duration of action as they appeared more potent than the reference antimir after 72 hours.

Also new bivalent antimirs 120, 121 and 122 had potency comparable to the reference antimir. Thus, bivalent antimirs with a linking moiety of nucleotides functions effectively.

1-11. (canceled)

12. A bivalent molecule comprising
a first oligonucleotide linked to a second oligonucleotide,
wherein the length of the first and the second oligonucleotide is between 7 and 12 nucleotides, and at least 50% of the nucleotides of the first and the second oligonucleotide is locked nucleic acid (LNA) monomers,
wherein the first and the second oligonucleotide comprise an antisense sequence complementary to a cellular mRNA or microRNA, and the antisense sequences act as steric blockers, wherein the first oligonucleotide and the second oligonucleotide comprise
i. a seed sequence of microRNA;
ii. a sequence capable of base pairing to the complementary sequence of a seed sequence; or
iii. a sequence capable of base pairing to a seed sequence, and
wherein the first and second oligonucleotide is linked via a linking moiety with a length of at least 10 angstrom;
wherein the linking moiety comprise a non-nucleotide polymer selected from the group consisting of: polyalkylen oxide, polyethyleneglycol, polyacrylic acid, polylactide acid (PLA), poly(glycolic acid) (PGA), polypropylene, polystyrene, polyolefin, polyamide, polycyanoacrylate, polyimide, polyethyleneterephtalat (PEY, PETG), polyethylene terephtalate (PETE), polytetramethylene glycol (PTG) and polyurethane; and
wherein the first and the second oligonucleotide is not an aptamer, siRNA, ribozyme, RNase H activating antisense oligonucleotide, full unmodified RNA oligonucleotide or full unmodified DNA oligonucleotide and is incapable of recruiting the RNAi machinery and incapable of activating RNase H.

13. The molecule of claim 12, wherein first and the second antisense sequence is a Blockmir antisense sequence capable of binding to a microRNA binding site in a target RNA, or wherein first and the second antisense sequence is an antimir antisense sequence capable of binding to a microRNA.

14. The molecule of claim 12 or 13, wherein the first and the second oligonucleotide comprise at least 75% LNA monomers.

15. The molecule of claim 12 or 13, wherein the linking moiety is incorporated as one or more monomers during standard oligonucleotide synthesis and wherein the monomer adapted for incorporation is selected from the group consisting of: Spacer 18 amidite (17-O-DMT-Hexaethyleneoxide-1-O-phosphoramidite), Spacer 9 Amidite (8-DMT-O-Triethyleneoxide-1-O-phosphoramidite), C6 Spacer Amidite (6-DMT-O-Hexanediol-1-O-Phosphoramidite) and C3 Spacer Amidite (DMT-1,3 propanediol-phosphoramidite).

16. The molecule of claim 14, wherein the linking moiety is incorporated as one or more monomers during standard oligonucleotide synthesis and wherein the monomer adapted for incorporation is selected from the group consisting of: Spacer 18 amidite (17-O-DMT-Hexaethyleneoxide-1-O-phosphoramidite), Spacer 9 Amidite (8-DMT-O-Triethyleneoxide-1-O-phosphoramidite), C6 Spacer Amidite (6-DMT-O-Hexanediol-1-O-Phosphoramidite) and C3 Spacer Amidite (DMT-1,3 propanediol-phosphoramidite).


SEQUENCE LISTING (723 sequences)

SEQ ID NO: 1 - RNA, 22 nt, Homo Sapiens
ugagguaguagguuguauaguu

SEQ ID NO: 2 - RNA, 21 nt, Homo Sapiens
cuauacaaucuacugucuuuc

SEQ ID NO: 3 - RNA, 22 nt, Homo Sapiens
ugagguaguagguugugugguu

SEQ ID NO: 4 - RNA, 22 nt, Homo Sapiens
cuauacaaccuacugccuuccc

SEQ ID NO: 5 - RNA, 22 nt, Homo Sapiens
ugagguaguagguuguaugguu

SEQ ID NO: 6 - RNA, 22 nt, Homo Sapiens
uagaguuacacccugggaguua

SEQ ID NO: 7 - RNA, 22 nt, Homo Sapiens
agagguaguagguugcauaguu

SEQ ID NO: 8 - RNA, 22 nt, Homo Sapiens
cuauacgaccugcugccuuucu

SEQ ID NO: 9 - RNA, 22 nt, Homo Sapiens
ugagguaggagguuguauaguu

SEQ ID NO: 10 - RNA, 22 nt, Homo Sapiens
cuauacggccuccuagcuuucc

SEQ ID NO: 11 - RNA, 22 nt, Homo Sapiens
ugagguaguagauuguauaguu

SEQ ID NO: 12 - RNA, 22 nt, Homo Sapiens
cuauacaaucuauugccuuccc

SEQ ID NO: 13 - RNA, 22 nt, Homo Sapiens
cuauacagucuacugucuuucc

SEQ ID NO: 14 - RNA, 22 nt, Homo Sapiens
ugagguaguaguuuguacaguu

SEQ ID NO: 15 - RNA, 21 nt, Homo Sapiens
cuguacaggccacugccuugc

SEQ ID NO: 16 - RNA, 22 nt, Homo Sapiens
ugagguaguaguuugugcuguu

SEQ ID NO: 17 - RNA, 22 nt, Homo Sapiens
cugcgcaagcuacugccuugcu

SEQ ID NO: 18 - RNA, 22 nt, Homo Sapiens
uggaauguaaagaaguauguau

SEQ ID NO: 19 - RNA, 22 nt, Homo Sapiens
aacccguagauccgaacuugug

SEQ ID NO: 20 - RNA, 22 nt, Homo Sapiens
caagcuuguaucuauagguaug

SEQ ID NO: 21 - RNA, 21 nt, Homo Sapiens
uacaguacugugauaacugaa

SEQ ID NO: 22 - RNA, 22 nt, Homo Sapiens
caguuaucacagugcugaugcu

SEQ ID NO: 23 - RNA, 23 nt, Homo Sapiens
agcagcauuguacagggcuauga

SEQ ID NO: 24 - RNA, 23 nt, Homo Sapiens
ucaaaugcucagacuccuguggu

SEQ ID NO: 25 - RNA, 22 nt, Homo Sapiens
acggauguuugagcaugugcua

SEQ ID NO: 26 - RNA, 23 nt, Homo Sapiens
aaaagugcuuacagugcagguag

SEQ ID NO: 27 - RNA, 22 nt, Homo Sapiens
cugcaauguaagcacuucuuac

SEQ ID NO: 28 - RNA, 21 nt, Homo Sapiens
uaaagugcugacagugcagau

SEQ ID NO: 29 - RNA, 22 nt, Homo Sapiens
ccgcacuguggguacuugcugc

SEQ ID NO: 30 - RNA, 23 nt, Homo Sapiens
agcagcauuguacagggcuauca

SEQ ID NO: 31 - RNA, 23 nt, Homo Sapiens
uacccuguagauccgaauuugug

SEQ ID NO: 32 - RNA, 22 nt, Homo Sapiens
caaauucguaucuaggggaaua

SEQ ID NO: 33 - RNA, 23 nt, Homo Sapiens
uacccuguagaaccgaauuugug

SEQ ID NO: 34 - RNA, 22 nt, Homo Sapiens
acagauucgauucuaggggaau

SEQ ID NO: 35 - RNA, 22 nt, Homo Sapiens
uggagugugacaaugguguuug

SEQ ID NO: 36 - RNA, 22 nt, Homo Sapiens
aacgccauuaucacacuaaaua

SEQ ID NO: 37 - RNA, 20 nt, Homo Sapiens
uaaggcacgcggugaaugcc

SEQ ID NO: 38 - RNA, 22 nt, Homo Sapiens
cguguucacagcggaccuugau

SEQ ID NO: 39 - RNA, 22 nt, Homo Sapiens
acaggugagguucuugggagcc

SEQ ID NO: 40 - RNA, 24 nt, Homo Sapiens
ucccugagacccuuuaaccuguga

SEQ ID NO: 41 - RNA, 22 nt, Homo Sapiens
ucccugagacccuaacuuguga

SEQ ID NO: 42 - RNA, 22 nt, Homo Sapiens
acggguuaggcucuugggagcu

SEQ ID NO: 43 - RNA, 22 nt, Homo Sapiens
ucacaagucaggcucuugggac

SEQ ID NO: 44 - RNA, 22 nt, Homo Sapiens
ucguaccgugaguaauaaugcg

SEQ ID NO: 45 - RNA, 21 nt, Homo Sapiens
cauuauuacuuuugguacgcg

SEQ ID NO: 46 - RNA, 22 nt, Homo Sapiens
ucggauccgucugagcuuggcu

SEQ ID NO: 47 - RNA, 22 nt, Homo Sapiens
cugaagcucagagggcucugau

SEQ ID NO: 48 - RNA, 21 nt, Homo Sapiens
ucacagugaaccggucucuuu

SEQ ID NO: 49 - RNA, 21 nt, Homo Sapiens
ucacagugaaccggucucuuu

SEQ ID NO: 50 - RNA, 22 nt, Homo Sapiens
aagcccuuaccccaaaaaguau

SEQ ID NO: 51 - RNA, 22 nt, Homo Sapiens
aagcccuuaccccaaaaagcau

SEQ ID NO: 52 - RNA, 21 nt, Homo Sapiens
cuuuuugcggucugggcuugc

SEQ ID NO: 53 - RNA, 22 nt, Homo Sapiens
cagugcaauguuaaaagggcau

SEQ ID NO: 54 - RNA, 22 nt, Homo Sapiens
uucacauugugcuacugucugc

SEQ ID NO: 55 - RNA, 22 nt, Homo Sapiens
cagugcaaugaugaaagggcau

SEQ ID NO: 56 - RNA, 21 nt, Homo Sapiens
acucuuucccuguugcacuac

SEQ ID NO: 57 - RNA, 22 nt, Homo Sapiens
uaacagucuacagccauggucg

SEQ ID NO: 58 - RNA, 22 nt, Homo Sapiens
accguggcuuucgauuguuacu

SEQ ID NO: 59 - RNA, 22 nt, Homo Sapiens
uuugguccccuucaaccagcug

SEQ ID NO: 60 - RNA, 22 nt, Homo Sapiens
uuugguccccuucaaccagcua

SEQ ID NO: 61 - RNA, 22 nt, Homo Sapiens
ugugacugguugaccagagggg

SEQ ID NO: 62 - RNA, 23 nt, Homo Sapiens
uauggcuuuuuauuccuauguga

SEQ ID NO: 63 - RNA, 22 nt, Homo Sapiens
uauagggauuggagccguggcg

SEQ ID NO: 64 - RNA, 23 nt, Homo Sapiens
uauggcuuuucauuccuauguga

SEQ ID NO: 65 - RNA, 22 nt, Homo Sapiens
auguagggcuaaaagccauggg

SEQ ID NO: 66 - RNA, 23 nt, Homo Sapiens
acuccauuuguuuugaugaugga

SEQ ID NO: 67 - RNA, 22 nt, Homo Sapiens
caucaucgucucaaaugagucu

SEQ ID NO: 68 - RNA, 23 nt, Homo Sapiens
uuauugcuuaagaauacgcguag

SEQ ID NO: 69 - RNA, 23 nt, Homo Sapiens
agcugguguugugaaucaggccg

SEQ ID NO: 70 - RNA, 22 nt, Homo Sapiens
gcuacuucacaacaccagggcc

SEQ ID NO: 71 - RNA, 22 nt, Homo Sapiens
gcuauuucacgacaccaggguu

SEQ ID NO: 72 - RNA, 22 nt, Homo Sapiens
ggagacgcggcccuguuggagu

SEQ ID NO: 73 - RNA, 22 nt, Homo Sapiens
ucuacagugcacgugucuccag

SEQ ID NO: 74 - RNA, 21 nt, Homo Sapiens
uaccacaggguagaaccacgg

SEQ ID NO: 75 - RNA, 22 nt, Homo Sapiens
cagugguuuuacccuaugguag

SEQ ID NO: 76 - RNA, 22 nt, Homo Sapiens
uaacacugucugguaaagaugg

SEQ ID NO: 77 - RNA, 22 nt, Homo Sapiens
caucuuccaguacaguguugga

SEQ ID NO: 78 - RNA, 23 nt, Homo Sapiens
uguaguguuuccuacuuuaugga

SEQ ID NO: 79 - RNA, 21 nt, Homo Sapiens
cauaaaguagaaagcacuacu

SEQ ID NO: 80 - RNA, 21 nt, Homo Sapiens
ugagaugaagcacuguagcuc

SEQ ID NO: 81 - RNA, 22 nt, Homo Sapiens
ggugcagugcugcaucucuggu

SEQ ID NO: 82 - RNA, 20 nt, Homo Sapiens
uacaguauagaugauguacu

SEQ ID NO: 83 - RNA, 22 nt, Homo Sapiens
ggauaucaucauauacuguaag

SEQ ID NO: 84 - RNA, 23 nt, Homo Sapiens
guccaguuuucccaggaaucccu

SEQ ID NO: 85 - RNA, 22 nt, Homo Sapiens
ggauuccuggaaauacuguucu

SEQ ID NO: 86 - RNA, 22 nt, Homo Sapiens
ugagaacugaauuccauggguu

SEQ ID NO: 87 - RNA, 22 nt, Homo Sapiens
ccucugaaauucaguucuucag

SEQ ID NO: 88 - RNA, 22 nt, Homo Sapiens
ugcccuguggacucaguucugg

SEQ ID NO: 89 - RNA, 22 nt, Homo Sapiens
ugagaacugaauuccauaggcu

SEQ ID NO: 90 - RNA, 20 nt, Homo Sapiens
guguguggaaaugcuucugc

SEQ ID NO: 91 - RNA, 22 nt, Homo Sapiens
gugugcggaaaugcuucugcua

SEQ ID NO: 92 - RNA, 22 nt, Homo Sapiens
ucagugcacuacagaacuuugu

SEQ ID NO: 93 - RNA, 22 nt, Homo Sapiens
aaaguucugagacacuccgacu

SEQ ID NO: 94 - RNA, 22 nt, Homo Sapiens
ucagugcaucacagaacuuugu

SEQ ID NO: 95 - RNA, 22 nt, Homo Sapiens
aaguucuguuauacacucaggc

SEQ ID NO: 96 - RNA, 23 nt, Homo Sapiens
ucuggcuccgugucuucacuccc

SEQ ID NO: 97 - RNA, 21 nt, Homo Sapiens
agggagggacgggggcugugc

SEQ ID NO: 98 - RNA, 22 nt, Homo Sapiens
ucucccaacccuuguaccagug

SEQ ID NO: 99 - RNA, 22 nt, Homo Sapiens
cugguacaggccugggggacag

SEQ ID NO: 100 - RNA, 21 nt, Homo Sapiens
cuagacugaagcuccuugagg

SEQ ID NO: 101 - RNA, 21 nt, Homo Sapiens
ucgaggagcucacagucuagu

SEQ ID NO: 102 - RNA, 21 nt, Homo Sapiens
ucagugcaugacagaacuugg

SEQ ID NO: 103 - RNA, 22 nt, Homo Sapiens
uugcauagucacaaaagugauc

SEQ ID NO: 104 - RNA, 22 nt, Homo Sapiens
uagguuauccguguugccuucg

SEQ ID NO: 105 - RNA, 22 nt, Homo Sapiens
aaucauacacgguugaccuauu

SEQ ID NO: 106 - RNA, 23 nt, Homo Sapiens
uuaaugcuaaucgugauaggggu

SEQ ID NO: 107 - RNA, 22 nt, Homo Sapiens
cuccuacauauuagcauuaaca

SEQ ID NO: 108 - RNA, 22 nt, Homo Sapiens
uagcagcacauaaugguuugug

SEQ ID NO: 109 - RNA, 22 nt, Homo Sapiens
caggccauauugugcugccuca

SEQ ID NO: 110 - RNA, 22 nt, Homo Sapiens
uagcagcacaucaugguuuaca

SEQ ID NO: 111 - RNA, 22 nt, Homo Sapiens
cgaaucauuauuugcugcucua

SEQ ID NO: 112 - RNA, 22 nt, Homo Sapiens
uagcagcacguaaauauuggcg

SEQ ID NO: 113 - RNA, 22 nt, Homo Sapiens
ccaguauuaacugugcugcuga

SEQ ID NO: 114 - RNA, 22 nt, Homo Sapiens
ccaauauuacugugcugcuuua

SEQ ID NO: 115 - RNA, 23 nt, Homo Sapiens
caaagugcuuacagugcagguag

SEQ ID NO: 116 - RNA, 22 nt, Homo Sapiens
acugcagugaaggcacuuguag

SEQ ID NO: 117 - RNA, 23 nt, Homo Sapiens
aacauucaacgcugucggugagu

SEQ ID NO: 118 - RNA, 22 nt, Homo Sapiens
accaucgaccguugauuguacc

SEQ ID NO: 119 - RNA, 22 nt, Homo Sapiens
accacugaccguugacuguacc

SEQ ID NO: 120 - RNA, 23 nt, Homo Sapiens
aacauucauugcugucggugggu

SEQ ID NO: 121 - RNA, 22 nt, Homo Sapiens
aacauucaaccugucggugagu

SEQ ID NO: 122 - RNA, 22 nt, Homo Sapiens
aaccaucgaccguugaguggac

SEQ ID NO: 123 - RNA, 23 nt, Homo Sapiens
aacauucauuguugucggugggu

SEQ ID NO: 124 - RNA, 24 nt, Homo Sapiens
uuuggcaaugguagaacucacacu

SEQ ID NO: 125 - RNA, 21 nt, Homo Sapiens
ugguucuagacuugccaacua

SEQ ID NO: 126 - RNA, 22 nt, Homo Sapiens
uauggcacugguagaauucacu

SEQ ID NO: 127 - RNA, 22 nt, Homo Sapiens
gugaauuaccgaagggccauaa

SEQ ID NO: 128 - RNA, 22 nt, Homo Sapiens
uggacggagaacugauaagggu

SEQ ID NO: 129 - RNA, 22 nt, Homo Sapiens
uggagagaaaggcaguuccuga

SEQ ID NO: 130 - RNA, 22 nt, Homo Sapiens
aggggcuggcuuuccucugguc

SEQ ID NO: 131 - RNA, 22 nt, Homo Sapiens
caaagaauucuccuuuugggcu

SEQ ID NO: 132 - RNA, 22 nt, Homo Sapiens
gcccaaaggugaauuuuuuggg

SEQ ID NO: 133 - RNA, 22 nt, Homo Sapiens
ucgugucuuguguugcagccgg

SEQ ID NO: 134 - RNA, 22 nt, Homo Sapiens
ggcuacaacacaggacccgggc

SEQ ID NO: 135 - RNA, 21 nt, Homo Sapiens
cucccacaugcaggguuugca

SEQ ID NO: 136 - RNA, 21 nt, Homo Sapiens
caucccuugcaugguggaggg

SEQ ID NO: 137 - RNA, 23 nt, Homo Sapiens
uaaggugcaucuagugcagauag

SEQ ID NO: 138 - RNA, 23 nt, Homo Sapiens
acugcccuaagugcuccuucugg

SEQ ID NO: 139 - RNA, 23 nt, Homo Sapiens
uaaggugcaucuagugcaguuag

SEQ ID NO: 140 - RNA, 22 nt, Homo Sapiens
ugcccuaaaugccccuucuggc

SEQ ID NO: 141 - RNA, 22 nt, Homo Sapiens
ugauauguuugauauauuaggu

SEQ ID NO: 142 - RNA, 21 nt, Homo Sapiens
ugauauguuugauauuggguu

SEQ ID NO: 143 - RNA, 23 nt, Homo Sapiens
caacggaaucccaaaagcagcug

SEQ ID NO: 144 - RNA, 22 nt, Homo Sapiens
gcugcgcuuggauuucgucccc

SEQ ID NO: 145 - RNA, 21 nt, Homo Sapiens
cugaccuaugaauugacagcc

SEQ ID NO: 146 - RNA, 22 nt, Homo Sapiens
cugccaauuccauaggucacag

SEQ ID NO: 147 - RNA, 22 nt, Homo Sapiens
aacuggccuacaaagucccagu

SEQ ID NO: 148 - RNA, 22 nt, Homo Sapiens
ugggucuuugcgggcgagauga

SEQ ID NO: 149 - RNA, 22 nt, Homo Sapiens
aacuggcccucaaagucccgcu

SEQ ID NO: 150 - RNA, 22 nt, Homo Sapiens
cgggguuuugagggcgagauga

SEQ ID NO: 151 - RNA, 22 nt, Homo Sapiens
uguaacagcaacuccaugugga

SEQ ID NO: 152 - RNA, 22 nt, Homo Sapiens
ccaguggggcugcuguuaucug

SEQ ID NO: 153 - RNA, 21 nt, Homo Sapiens
uagcagcacagaaauauuggc

SEQ ID NO: 154 - RNA, 22 nt, Homo Sapiens
ccaauauuggcugugcugcucc

SEQ ID NO: 155 - RNA, 22 nt, Homo Sapiens
uagguaguuucauguuguuggg

SEQ ID NO: 156 - RNA, 22 nt, Homo Sapiens
cggcaacaagaaacugccugag

SEQ ID NO: 157 - RNA, 22 nt, Homo Sapiens
uagguaguuuccuguuguuggg

SEQ ID NO: 158 - RNA, 22 nt, Homo Sapiens
uucaccaccuucuccacccagc

SEQ ID NO: 159 - RNA, 22 nt, Homo Sapiens
gguccagaggggagauagguuc

SEQ ID NO: 160 - RNA, 22 nt, Homo Sapiens
acaguagucugcacauugguua

SEQ ID NO: 161 - RNA, 23 nt, Homo Sapiens
cccaguguucagacuaccuguuc

SEQ ID NO: 162 - RNA, 22 nt, Homo Sapiens
acaguagucugcacauugguua

SEQ ID NO: 163 - RNA, 23 nt, Homo Sapiens
cccaguguuuagacuaucuguuc

SEQ ID NO: 164 - RNA, 23 nt, Homo Sapiens
ugugcaaaucuaugcaaaacuga

SEQ ID NO: 165 - RNA, 22 nt, Homo Sapiens
aguuuugcauaguugcacuaca

SEQ ID NO: 166 - RNA, 23 nt, Homo Sapiens
ugugcaaauccaugcaaaacuga

SEQ ID NO: 167 - RNA, 23 nt, Homo Sapiens
aguuuugcagguuugcauccagc

SEQ ID NO: 168 - RNA, 22 nt, Homo Sapiens
aguuuugcagguuugcauuuca

SEQ ID NO: 169 - RNA, 22 nt, Homo Sapiens
uaacacugucugguaacgaugu

SEQ ID NO: 170 - RNA, 22 nt, Homo Sapiens
caucuuaccggacagugcugga

SEQ ID NO: 171 - RNA, 22 nt, Homo Sapiens
uaauacugccugguaaugauga

SEQ ID NO: 172 - RNA, 22 nt, Homo Sapiens
caucuuacugggcagcauugga

SEQ ID NO: 173 - RNA, 23 nt, Homo Sapiens
uaauacugccggguaaugaugga

SEQ ID NO: 174 - RNA, 22 nt, Homo Sapiens
cgucuuacccagcaguguuugg

SEQ ID NO: 175 - RNA, 20 nt, Homo Sapiens
agagguauagggcaugggaa

SEQ ID NO: 176 - RNA, 22 nt, Homo Sapiens
uuccuaugcauauacuucuuug

SEQ ID NO: 177 - RNA, 22 nt, Homo Sapiens
gugaaauguuuaggaccacuag

SEQ ID NO: 178 - RNA, 22 nt, Homo Sapiens
uucccuuugucauccuaugccu

SEQ ID NO: 179 - RNA, 22 nt, Homo Sapiens
uccuucauuccaccggagucug

SEQ ID NO: 180 - RNA, 22 nt, Homo Sapiens
uggaauguaaggaagugugugg

SEQ ID NO: 181 - RNA, 22 nt, Homo Sapiens
auaagacgagcaaaaagcuugu

SEQ ID NO: 182 - RNA, 22 nt, Homo Sapiens
auaagacgaacaaaagguuugu

SEQ ID NO: 183 - RNA, 23 nt, Homo Sapiens
uaaagugcuuauagugcagguag

SEQ ID NO: 184 - RNA, 22 nt, Homo Sapiens
acugcauuaugagcacuuaaag

SEQ ID NO: 185 - RNA, 23 nt, Homo Sapiens
caaagugcucauagugcagguag

SEQ ID NO: 186 - RNA, 22 nt, Homo Sapiens
acuguaguaugggcacuuccag

SEQ ID NO: 187 - RNA, 22 nt, Homo Sapiens
uagcuuaucagacugauguuga

SEQ ID NO: 188 - RNA, 21 nt, Homo Sapiens
caacaccagucgaugggcugu

SEQ ID NO: 189 - RNA, 22 nt, Homo Sapiens
cugugcgugugacagcggcuga

SEQ ID NO: 190 - RNA, 22 nt, Homo Sapiens
uucccuuugucauccuucgccu

SEQ ID NO: 191 - RNA, 21 nt, Homo Sapiens
uaacagucuccagucacggcc

SEQ ID NO: 192 - RNA, 22 nt, Homo Sapiens
acagcaggcacagacaggcagu

SEQ ID NO: 193 - RNA, 22 nt, Homo Sapiens
ugccugucuacacuugcugugc

SEQ ID NO: 194 - RNA, 21 nt, Homo Sapiens
augaccuaugaauugacagac

SEQ ID NO: 195 - RNA, 22 nt, Homo Sapiens
uaaucucagcuggcaacuguga

SEQ ID NO: 196 - RNA, 22 nt, Homo Sapiens
aaaucucugcaggcaaauguga

SEQ ID NO: 197 - RNA, 23 nt, Homo Sapiens
uacugcaucaggaacugauugga

SEQ ID NO: 198 - RNA, 21 nt, Homo Sapiens
uugugcuugaucuaaccaugu

SEQ ID NO: 199 - RNA, 22 nt, Homo Sapiens
augguuccgucaagcaccaugg

SEQ ID NO: 200 - RNA, 22 nt, Homo Sapiens
caugguucugucaagcaccgcg

SEQ ID NO: 201 - RNA, 22 nt, Homo Sapiens
agaguugagucuggacgucccg

SEQ ID NO: 202 - RNA, 22 nt, Homo Sapiens
agaauuguggcuggacaucugu

SEQ ID NO: 203 - RNA, 21 nt, Homo Sapiens
ugauuguccaaacgcaauucu

SEQ ID NO: 204 - RNA, 22 nt, Homo Sapiens
aagcugccaguugaagaacugu

SEQ ID NO: 205 - RNA, 22 nt, Homo Sapiens
aguucuucaguggcaagcuuua

SEQ ID NO: 206 - RNA, 21 nt, Homo Sapiens
ccacaccguaucugacacuuu

SEQ ID NO: 207 - RNA, 21 nt, Homo Sapiens
ccaccaccgugucugacacuu

SEQ ID NO: 208 - RNA, 22 nt, Homo Sapiens
acacagggcuguugugaagacu

SEQ ID NO: 209 - RNA, 23 nt, Homo Sapiens
agcuacauugucugcuggguuuc

SEQ ID NO: 210 - RNA, 22 nt, Homo Sapiens
accuggcauacaauguagauuu

SEQ ID NO: 211 - RNA, 21 nt, Homo Sapiens
agcuacaucuggcuacugggu

SEQ ID NO: 212 - RNA, 22 nt, Homo Sapiens
cucaguagccaguguagauccu

SEQ ID NO: 213 - RNA, 22 nt, Homo Sapiens
ugucaguuugucaaauacccca

SEQ ID NO: 214 - RNA, 22 nt, Homo Sapiens
cguguauuugacaagcugaguu

SEQ ID NO: 215 - RNA, 21 nt, Homo Sapiens
caagucacuagugguuccguu

SEQ ID NO: 216 - RNA, 21 nt, Homo Sapiens
aucacauugccagggauuucc

SEQ ID NO: 217 - RNA, 22 nt, Homo Sapiens
gggguuccuggggaugggauuu

SEQ ID NO: 218 - RNA, 21 nt, Homo Sapiens
aucacauugccagggauuacc

SEQ ID NO: 219 - RNA, 22 nt, Homo Sapiens
uggguuccuggcaugcugauuu

SEQ ID NO: 220 - RNA, 22 nt, Homo Sapiens
uggcucaguucagcaggaacag

SEQ ID NO: 221 - RNA, 22 nt, Homo Sapiens
ugccuacugagcugauaucagu

SEQ ID NO: 222 - RNA, 22 nt, Homo Sapiens
ugccuacugagcugaaacacag

SEQ ID NO: 223 - RNA, 22 nt, Homo Sapiens
cauugcacuugucucggucuga

SEQ ID NO: 224 - RNA, 21 nt, Homo Sapiens
aggcggagacuugggcaauug

SEQ ID NO: 225 - RNA, 22 nt, Homo Sapiens
uucaaguaauccaggauaggcu

SEQ ID NO: 226 - RNA, 22 nt, Homo Sapiens
ccuauucuugguuacuugcacg

SEQ ID NO: 227 - RNA, 22 nt, Homo Sapiens
ccuauucuugauuacuuguuuc

SEQ ID NO: 228 - RNA, 21 nt, Homo Sapiens
uucaaguaauucaggauaggu

SEQ ID NO: 229 - RNA, 22 nt, Homo Sapiens
ccuguucuccauuacuuggcuc

SEQ ID NO: 230 - RNA, 21 nt, Homo Sapiens
uucacaguggcuaaguuccgc

SEQ ID NO: 231 - RNA, 22 nt, Homo Sapiens
agggcuuagcugcuugugagca

SEQ ID NO: 232 - RNA, 21 nt, Homo Sapiens
uucacaguggcuaaguucugc

SEQ ID NO: 233 - RNA, 22 nt, Homo Sapiens
agagcuuagcugauuggugaac

SEQ ID NO: 234 - RNA, 22 nt, Homo Sapiens
cacuagauugugagcuccugga

SEQ ID NO: 235 - RNA, 22 nt, Homo Sapiens
aaggagcucacagucuauugag

SEQ ID NO: 236 - RNA, 22 nt, Homo Sapiens
gaggguuggguggaggcucucc

SEQ ID NO: 237 - RNA, 21 nt, Homo Sapiens
agggcccccccucaauccugu

SEQ ID NO: 238 - RNA, 21 nt, Homo Sapiens
auguaugugugcaugugcaug

SEQ ID NO: 239 - RNA, 24 nt, Homo Sapiens
agcagaagcagggagguucuccca

SEQ ID NO: 240 - RNA, 22 nt, Homo Sapiens
uaugugggaugguaaaccgcuu

SEQ ID NO: 241 - RNA, 22 nt, Homo Sapiens
ugguuuaccgucccacauacau

SEQ ID NO: 242 - RNA, 22 nt, Homo Sapiens
uagcaccaucugaaaucgguua

SEQ ID NO: 243 - RNA, 22 nt, Homo Sapiens
acugauuucuuuugguguucag

SEQ ID NO: 244 - RNA, 23 nt, Homo Sapiens
uagcaccauuugaaaucaguguu

SEQ ID NO: 245 - RNA, 24 nt, Homo Sapiens
gcugguuucauauggugguuuaga

SEQ ID NO: 246 - RNA, 22 nt, Homo Sapiens
cugguuucacaugguggcuuag

SEQ ID NO: 247 - RNA, 22 nt, Homo Sapiens
uagcaccauuugaaaucgguua

SEQ ID NO: 248 - RNA, 22 nt, Homo Sapiens
ugaccgauuucuccugguguuc

SEQ ID NO: 249 - RNA, 22 nt, Homo Sapiens
uauacaagggcagacucucucu

SEQ ID NO: 250 - RNA, 23 nt, Homo Sapiens
cagugcaauaguauugucaaagc

SEQ ID NO: 251 - RNA, 23 nt, Homo Sapiens
cagugcaaugauauugucaaagc

SEQ ID NO: 252 - RNA, 23 nt, Homo Sapiens
uaagugcuuccauguuuugguga

SEQ ID NO: 253 - RNA, 23 nt, Homo Sapiens
acuuaaacguggauguacuugcu

SEQ ID NO: 254 - RNA, 23 nt, Homo Sapiens
uaagugcuuccauguuuuaguag

SEQ ID NO: 255 - RNA, 22 nt, Homo Sapiens
acuuuaacauggaagugcuuuc

SEQ ID NO: 256 - RNA, 23 nt, Homo Sapiens
uaagugcuuccauguuucagugg

SEQ ID NO: 257 - RNA, 22 nt, Homo Sapiens
uuuaacauggggguaccugcug

SEQ ID NO: 258 - RNA, 23 nt, Homo Sapiens
uaagugcuuccauguuugagugu

SEQ ID NO: 259 - RNA, 22 nt, Homo Sapiens
acuuuaacauggaggcacuugc

SEQ ID NO: 260 - RNA, 22 nt, Homo Sapiens
uguaaacauccucgacuggaag

SEQ ID NO: 261 - RNA, 22 nt, Homo Sapiens
cuuucagucggauguuugcagc

SEQ ID NO: 262 - RNA, 22 nt, Homo Sapiens
uguaaacauccuacacucagcu

SEQ ID NO: 263 - RNA, 22 nt, Homo Sapiens
cugggagguggauguuuacuuc

SEQ ID NO: 264 - RNA, 23 nt, Homo Sapiens
uguaaacauccuacacucucagc

SEQ ID NO: 265 - RNA, 22 nt, Homo Sapiens
cugggagaggguuguuuacucc

SEQ ID NO: 266 - RNA, 22 nt, Homo Sapiens
cugggagaaggcuguuuacucu

SEQ ID NO: 267 - RNA, 22 nt, Homo Sapiens
uguaaacauccccgacuggaag

SEQ ID NO: 268 - RNA, 22 nt, Homo Sapiens
cuuucagucagauguuugcugc

SEQ ID NO: 269 - RNA, 22 nt, Homo Sapiens
uguaaacauccuugacuggaag

SEQ ID NO: 270 - RNA, 22 nt, Homo Sapiens
cuuucagucggauguuuacagc

SEQ ID NO: 271 - RNA, 21 nt, Homo Sapiens
aggcaagaugcuggcauagcu

SEQ ID NO: 272 - RNA, 22 nt, Homo Sapiens
ugcuaugccaacauauugccau

SEQ ID NO: 273 - RNA, 22 nt, Homo Sapiens
uauugcacauuacuaaguugca

SEQ ID NO: 274 - RNA, 22 nt, Homo Sapiens
caauuuagugugugugauauuu

SEQ ID NO: 275 - RNA, 22 nt, Homo Sapiens
aaaagcuggguugagagggcga

SEQ ID NO: 276 - RNA, 21 nt, Homo Sapiens
cacauuacacggucgaccucu

SEQ ID NO: 277 - RNA, 22 nt, Homo Sapiens
aggugguccguggcgcguucgc

SEQ ID NO: 278 - RNA, 20 nt, Homo Sapiens
acugccccaggugcugcugg

SEQ ID NO: 279 - RNA, 23 nt, Homo Sapiens
cgcauccccuagggcauuggugu

SEQ ID NO: 280 - RNA, 23 nt, Homo Sapiens
ccuaguagguguccaguaagugu

SEQ ID NO: 281 - RNA, 20 nt, Homo Sapiens
ccucugggcccuuccuccag

SEQ ID NO: 282 - RNA, 22 nt, Homo Sapiens
cuggcccucucugcccuuccgu

SEQ ID NO: 283 - RNA, 22 nt, Homo Sapiens
aacacaccugguuaaccucuuu

SEQ ID NO: 284 - RNA, 23 nt, Homo Sapiens
gcaaagcacacggccugcagaga

SEQ ID NO: 285 - RNA, 22 nt, Homo Sapiens
ucucugggccugugucuuaggc

SEQ ID NO: 286 - RNA, 21 nt, Homo Sapiens
gccccugggccuauccuagaa

SEQ ID NO: 287 - RNA, 22 nt, Homo Sapiens
cuagguauggucccagggaucc

SEQ ID NO: 288 - RNA, 23 nt, Homo Sapiens
ucaagagcaauaacgaaaaaugu

SEQ ID NO: 289 - RNA, 22 nt, Homo Sapiens
uuuuucauuauugcuccugacc

SEQ ID NO: 290 - RNA, 22 nt, Homo Sapiens
cuccuauaugaugccuuucuuc

SEQ ID NO: 291 - RNA, 21 nt, Homo Sapiens
gaacggcuucauacaggaguu

SEQ ID NO: 292 - RNA, 22 nt, Homo Sapiens
uccagcaucagugauuuuguug

SEQ ID NO: 293 - RNA, 22 nt, Homo Sapiens
aacaauauccuggugcugagug

SEQ ID NO: 294 - RNA, 23 nt, Homo Sapiens
ugagcgccucgacgacagagccg

SEQ ID NO: 295 - RNA, 23 nt, Homo Sapiens
ucccuguccuccaggagcucacg

SEQ ID NO: 296 - RNA, 21 nt, Homo Sapiens
gugcauuguaguugcauugca

SEQ ID NO: 297 - RNA, 22 nt, Homo Sapiens
caauguuuccacagugcaucac

SEQ ID NO: 298 - RNA, 20 nt, Homo Sapiens
gugcauugcuguugcauugc

SEQ ID NO: 299 - RNA, 22 nt, Homo Sapiens
cagugccucggcagugcagccc

SEQ ID NO: 300 - RNA, 22 nt, Homo Sapiens
uuauaaagcaaugagacugauu

SEQ ID NO: 301 - RNA, 22 nt, Homo Sapiens
uccgucucaguuacuuuauagc

SEQ ID NO: 302 - RNA, 23 nt, Homo Sapiens
ucucacacagaaaucgcacccgu

SEQ ID NO: 303 - RNA, 21 nt, Homo Sapiens
aggggugcuaucugugauuga

SEQ ID NO: 304 - RNA, 22 nt, Homo Sapiens
gcugacuccuaguccagggcuc

SEQ ID NO: 305 - RNA, 23 nt, Homo Sapiens
ugucugcccgcaugccugccucu

SEQ ID NO: 306 - RNA, 22 nt, Homo Sapiens
uggcagugucuuagcugguugu

SEQ ID NO: 307 - RNA, 22 nt, Homo Sapiens
caaucagcaaguauacugcccu

SEQ ID NO: 308 - RNA, 22 nt, Homo Sapiens
caaucacuaacuccacugccau

SEQ ID NO: 309 - RNA, 23 nt, Homo Sapiens
uaggcagugucauuagcugauug

SEQ ID NO: 310 - RNA, 22 nt, Homo Sapiens
aaucacuaaccacacggccagg

SEQ ID NO: 311 - RNA, 23 nt, Homo Sapiens
aggcaguguaguuagcugauugc

SEQ ID NO: 312 - RNA, 23 nt, Homo Sapiens
ucccccaggugugauucugauuu

SEQ ID NO: 313 - RNA, 22 nt, Homo Sapiens
uuaucagaaucuccagggguac

SEQ ID NO: 314 - RNA, 22 nt, Homo Sapiens
aacacaccuauucaaggauuca

SEQ ID NO: 315 - RNA, 24 nt, Homo Sapiens
aauccuuggaaccuaggugugagu

SEQ ID NO: 316 - RNA, 22 nt, Homo Sapiens
aauugcacgguauccaucugua

SEQ ID NO: 317 - RNA, 22 nt, Homo Sapiens
cggguggaucacgaugcaauuu

SEQ ID NO: 318 - RNA, 22 nt, Homo Sapiens
uaaugccccuaaaaauccuuau

SEQ ID NO: 319 - RNA, 22 nt, Homo Sapiens
aauugcacuuuagcaaugguga

SEQ ID NO: 320 - RNA, 22 nt, Homo Sapiens
acuguugcuaauaugcaacucu

SEQ ID NO: 321 - RNA, 21 nt, Homo Sapiens
aauaauacaugguugaucuuu

SEQ ID NO: 322 - RNA, 22 nt, Homo Sapiens
agaucgaccguguuauauucgc

SEQ ID NO: 323 - RNA, 22 nt, Homo Sapiens
gccugcugggguggaaccuggu

SEQ ID NO: 324 - RNA, 23 nt, Homo Sapiens
aagugccgccaucuuuugagugu

SEQ ID NO: 325 - RNA, 20 nt, Homo Sapiens
acucaaacugugggggcacu

SEQ ID NO: 326 - RNA, 23 nt, Homo Sapiens
aaagugcugcgacauuugagcgu

SEQ ID NO: 327 - RNA, 23 nt, Homo Sapiens
gaagugcuucgauuuuggggugu

SEQ ID NO: 328 - RNA, 22 nt, Homo Sapiens
acucaaaaugggggcgcuuucc

SEQ ID NO: 329 - RNA, 22 nt, Homo Sapiens
uuauaauacaaccugauaagug

SEQ ID NO: 330 - RNA, 22 nt, Homo Sapiens
cuuaucagauuguauuguaauu

SEQ ID NO: 331 - RNA, 22 nt, Homo Sapiens
auauaauacaaccugcuaagug

SEQ ID NO: 332 - RNA, 22 nt, Homo Sapiens
cuuagcagguuguauuaucauu

SEQ ID NO: 333 - RNA, 22 nt, Homo Sapiens
uuuguucguucggcucgcguga

SEQ ID NO: 334 - RNA, 21 nt, Homo Sapiens
aucauagaggaaaauccacgu

SEQ ID NO: 335 - RNA, 22 nt, Homo Sapiens
guagauucuccuucuaugagua

SEQ ID NO: 336 - RNA, 22 nt, Homo Sapiens
aucauagaggaaaauccauguu

SEQ ID NO: 337 - RNA, 21 nt, Homo Sapiens
aacauagaggaaauuccacgu

SEQ ID NO: 338 - RNA, 22 nt, Homo Sapiens
aucacacaaaggcaacuuuugu

SEQ ID NO: 339 - RNA, 22 nt, Homo Sapiens
agagguugcccuuggugaauuc

SEQ ID NO: 340 - RNA, 21 nt, Homo Sapiens
acuggacuuggagucagaagg

SEQ ID NO: 341 - RNA, 22 nt, Homo Sapiens
cuccugacuccagguccugugu

SEQ ID NO: 342 - RNA, 21 nt, Homo Sapiens
ugguagacuauggaacguagg

SEQ ID NO: 343 - RNA, 22 nt, Homo Sapiens
uauguaacaugguccacuaacu

SEQ ID NO: 344 - RNA, 22 nt, Homo Sapiens
uauguaauaugguccacaucuu

SEQ ID NO: 345 - RNA, 22 nt, Homo Sapiens
ugguugaccauagaacaugcgc

SEQ ID NO: 346 - RNA, 22 nt, Homo Sapiens
uauacaagggcaagcucucugu

SEQ ID NO: 347 - RNA, 22 nt, Homo Sapiens
gaaguuguucgugguggauucg

SEQ ID NO: 348 - RNA, 22 nt, Homo Sapiens
agaucagaaggugauuguggcu

SEQ ID NO: 349 - RNA, 20 nt, Homo Sapiens
auuccuagaaauuguucaua

SEQ ID NO: 350 - RNA, 22 nt, Homo Sapiens
gaauguugcucggugaaccccu

SEQ ID NO: 351 - RNA, 23 nt, Homo Sapiens
agguuacccgagcaacuuugcau

SEQ ID NO: 352 - RNA, 21 nt, Homo Sapiens
aauauaacacagauggccugu

SEQ ID NO: 353 - RNA, 21 nt, Homo Sapiens
uaguagaccguauagcguacg

SEQ ID NO: 354 - RNA, 22 nt, Homo Sapiens
uauguaacacgguccacuaacc

SEQ ID NO: 355 - RNA, 23 nt, Homo Sapiens
acuucaccugguccacuagccgu

SEQ ID NO: 356 - RNA, 23 nt, Homo Sapiens
aucaacagacauuaauugggcgc

SEQ ID NO: 357 - RNA, 22 nt, Homo Sapiens
acuggacuuagggucagaaggc

SEQ ID NO: 358 - RNA, 23 nt, Homo Sapiens
agcucggucugaggccccucagu

SEQ ID NO: 359 - RNA, 23 nt, Homo Sapiens
ugaggggcagagagcgagacuuu

SEQ ID NO: 360 - RNA, 22 nt, Homo Sapiens
cagcagcaauucauguuuugaa

SEQ ID NO: 361 - RNA, 21 nt, Homo Sapiens
caaaacgugaggcgcugcuau

SEQ ID NO: 362 - RNA, 23 nt, Homo Sapiens
aaugacacgaucacucccguuga

SEQ ID NO: 363 - RNA, 22 nt, Homo Sapiens
aucgggaaugucguguccgccc

SEQ ID NO: 364 - RNA, 22 nt, Homo Sapiens
uaauacugucugguaaaaccgu

SEQ ID NO: 365 - RNA, 21 nt, Homo Sapiens
ugucuugcaggccgucaugca

SEQ ID NO: 366 - RNA, 22 nt, Homo Sapiens
caggucgucuugcagggcuucu

SEQ ID NO: 367 - RNA, 23 nt, Homo Sapiens
ucuuggaguaggucauugggugg

SEQ ID NO: 368 - RNA, 21 nt, Homo Sapiens
cuggauggcuccuccaugucu

SEQ ID NO: 369 - RNA, 22 nt, Homo Sapiens
aucaugaugggcuccucggugu

SEQ ID NO: 370 - RNA, 22 nt, Homo Sapiens
uugcauauguaggaugucccau

SEQ ID NO: 371 - RNA, 22 nt, Homo Sapiens
uggcaguguauuguuagcuggu

SEQ ID NO: 372 - RNA, 22 nt, Homo Sapiens
aggcaguguauuguuagcuggc

SEQ ID NO: 373 - RNA, 22 nt, Homo Sapiens
uuuugcgauguguuccuaauau

SEQ ID NO: 374 - RNA, 22 nt, Homo Sapiens
uugggaucauuuugcauccaua

SEQ ID NO: 375 - RNA, 22 nt, Homo Sapiens
uuuugcaauauguuccugaaua

SEQ ID NO: 376 - RNA, 22 nt, Homo Sapiens
aaaccguuaccauuacugaguu

SEQ ID NO: 377 - RNA, 22 nt, Homo Sapiens
aacuguuugcagaggaaacuga

SEQ ID NO: 378 - RNA, 22 nt, Homo Sapiens
cucaucugcaaagaaguaagug

SEQ ID NO: 379 - RNA, 23 nt, Homo Sapiens
agguuguccguggugaguucgca

SEQ ID NO: 380 - RNA, 23 nt, Homo Sapiens
uagugcaauauugcuuauagggu

SEQ ID NO: 381 - RNA, 22 nt, Homo Sapiens
acccuaucaauauugucucugc

SEQ ID NO: 382 - RNA, 21 nt, Homo Sapiens
gcaguccaugggcauauacac

SEQ ID NO: 383 - RNA, 22 nt, Homo Sapiens
uaugugccuuuggacuacaucg

SEQ ID NO: 384 - RNA, 21 nt, Homo Sapiens
ucacuccucuccucccgucuu

SEQ ID NO: 385 - RNA, 22 nt, Homo Sapiens
aagacgggaggaaagaagggag

SEQ ID NO: 386 - RNA, 22 nt, Homo Sapiens
ucaggcucaguccccucccgau

SEQ ID NO: 387 - RNA, 22 nt, Homo Sapiens
gucauacacggcucuccucucu

SEQ ID NO: 388 - RNA, 22 nt, Homo Sapiens
agaggcuggccgugaugaauuc

SEQ ID NO: 389 - RNA, 21 nt, Homo Sapiens
cggggcagcucaguacaggau

SEQ ID NO: 390 - RNA, 22 nt, Homo Sapiens
uccuguacugagcugccccgag

SEQ ID NO: 391 - RNA, 22 nt, Homo Sapiens
aaucauacagggacauccaguu

SEQ ID NO: 392 - RNA, 22 nt, Homo Sapiens
aaucguacagggucauccacuu

SEQ ID NO: 393 - RNA, 21 nt, Homo Sapiens
uugaaaggcuauuucuugguc

SEQ ID NO: 394 - RNA, 21 nt, Homo Sapiens
cccagauaauggcacucucaa

SEQ ID NO: 395 - RNA, 22 nt, Homo Sapiens
gugacaucacauauacggcagc

SEQ ID NO: 396 - RNA, 22 nt, Homo Sapiens
caaccuggaggacuccaugcug

SEQ ID NO: 397 - RNA, 20 nt, Homo Sapiens
ccauggaucuccaggugggu

SEQ ID NO: 398 - RNA, 22 nt, Homo Sapiens
cuuaugcaagauucccuucuac

SEQ ID NO: 399 - RNA, 22 nt, Homo Sapiens
aguggggaacccuuccaugagg

SEQ ID NO: 400 - RNA, 23 nt, Homo Sapiens
aggaccugcgggacaagauucuu

SEQ ID NO: 401 - RNA, 22 nt, Homo Sapiens
ugaaggucuacugugugccagg

SEQ ID NO: 402 - RNA, 22 nt, Homo Sapiens
uuguacaugguaggcuuucauu

SEQ ID NO: 403 - RNA, 22 nt, Homo Sapiens
ugaaacauacacgggaaaccuc

SEQ ID NO: 404 - RNA, 22 nt, Homo Sapiens
aaacaaacauggugcacuucuu

SEQ ID NO: 405 - RNA, 22 nt, Homo Sapiens
ugaguauuacauggccaaucuc

SEQ ID NO: 406 - RNA, 21 nt, Homo Sapiens
cagcagcacacugugguuugu

SEQ ID NO: 407 - RNA, 22 nt, Homo Sapiens
caaaccacacugugguguuaga

SEQ ID NO: 408 - RNA, 23 nt, Homo Sapiens
uuucaagccagggggcguuuuuc

SEQ ID NO: 409 - RNA, 22 nt, Homo Sapiens
aacaucacagcaagucugugcu

SEQ ID NO: 410 - RNA, 21 nt, Homo Sapiens
uuaagacuugcagugauguuu

SEQ ID NO: 411 - RNA, 23 nt, Homo Sapiens
uaauccuugcuaccugggugaga

SEQ ID NO: 412 - RNA, 22 nt, Homo Sapiens
augcaccugggcaaggauucug

SEQ ID NO: 413 - RNA, 22 nt, Homo Sapiens
aaugcacccgggcaaggauucu

SEQ ID NO: 414 - RNA, 22 nt, Homo Sapiens
aauccuuugucccugggugaga

SEQ ID NO: 415 - RNA, 22 nt, Homo Sapiens
aaugcaccugggcaaggauuca

SEQ ID NO: 416 - RNA, 21 nt, Homo Sapiens
auccuugcuaucugggugcua

SEQ ID NO: 417 - RNA, 23 nt, Homo Sapiens
uagcagcgggaacaguucugcag

SEQ ID NO: 418 - RNA, 22 nt, Homo Sapiens
agacccuggucugcacucuauc

SEQ ID NO: 419 - RNA, 22 nt, Homo Sapiens
cgucaacacuugcugguuuccu

SEQ ID NO: 420 - RNA, 22 nt, Homo Sapiens
gggagccaggaaguauugaugu

SEQ ID NO: 421 - RNA, 21 nt, Homo Sapiens
uaaggcacccuucugaguaga

SEQ ID NO: 422 - RNA, 21 nt, Homo Sapiens
uuuugcaccuuuuggagugaa

SEQ ID NO: 423 - RNA, 23 nt, Homo Sapiens
ugauuguagccuuuuggaguaga

SEQ ID NO: 424 - RNA, 23 nt, Homo Sapiens
uacuccagagggcgucacucaug

SEQ ID NO: 425 - RNA, 22 nt, Homo Sapiens
uacugcagacguggcaaucaug

SEQ ID NO: 426 - RNA, 22 nt, Homo Sapiens
ugauugguacgucuguggguag

SEQ ID NO: 427 - RNA, 21 nt, Homo Sapiens
uacugcagacaguggcaauca

SEQ ID NO: 428 - RNA, 22 nt, Homo Sapiens
uacucaggagaguggcaaucac

SEQ ID NO: 429 - RNA, 21 nt, Homo Sapiens
gugucuuuugcucugcaguca

SEQ ID NO: 430 - RNA, 22 nt, Homo Sapiens
aagugcugucauagcugagguc

SEQ ID NO: 431 - RNA, 23 nt, Homo Sapiens
cacucagccuugagggcacuuuc

SEQ ID NO: 432 - RNA, 23 nt, Homo Sapiens
uaaauuucaccuuucugagaagg

SEQ ID NO: 433 - RNA, 18 nt, Homo Sapiens
uucacagggaggugucau

SEQ ID NO: 434 - RNA, 21 nt, Homo Sapiens
auugacacuucugugaguaga

SEQ ID NO: 435 - RNA, 22 nt, Homo Sapiens
gagugccuucuuuuggagcguu

SEQ ID NO: 436 - RNA, 24 nt, Homo Sapiens
uucuccaaaagaaagcacuuucug

SEQ ID NO: 437 - RNA, 18 nt, Homo Sapiens
ugcuuccuuucagagggu

SEQ ID NO: 438 - RNA, 23 nt, Homo Sapiens
uucucgaggaaagaagcacuuuc

SEQ ID NO: 439 - RNA, 22 nt, Homo Sapiens
aucuggagguaagaagcacuuu

SEQ ID NO: 440 - RNA, 18 nt, Homo Sapiens
ugcuuccuuucagagggu

SEQ ID NO: 441 - RNA, 22 nt, Homo Sapiens
ccucuagauggaagcacugucu

SEQ ID NO: 442 - RNA, 22 nt, Homo Sapiens
aucgugcaucccuuuagagugu

SEQ ID NO: 443 - RNA, 22 nt, Homo Sapiens
ucgugcaucccuuuagaguguu

SEQ ID NO: 444 - RNA, 22 nt, Homo Sapiens
aucgugcauccuuuuagagugu

SEQ ID NO: 445 - RNA, 22 nt, Homo Sapiens
gaaagcgcuucccuuugcugga

SEQ ID NO: 446 - RNA, 20 nt, Homo Sapiens
cugcaaagggaagcccuuuc

SEQ ID NO: 447 - RNA, 22 nt, Homo Sapiens
caaagcgcuccccuuuagaggu

SEQ ID NO: 448 - RNA, 23 nt, Homo Sapiens
caaagcgcuucucuuuagagugu

SEQ ID NO: 449 - RNA, 23 nt, Homo Sapiens
ucucuggagggaagcacuuucug

SEQ ID NO: 450 - RNA, 21 nt, Homo Sapiens
caaagcgcuucccuuuggagc

SEQ ID NO: 451 - RNA, 22 nt, Homo Sapiens
cucuagagggaagcacuuucug

SEQ ID NO: 452 - RNA, 21 nt, Homo Sapiens
aaagcgcuucccuucagagug

SEQ ID NO: 453 - RNA, 22 nt, Homo Sapiens
cucuagagggaagcgcuuucug

SEQ ID NO: 454 - RNA, 21 nt, Homo Sapiens
gaaagcgcuucucuuuagagg

SEQ ID NO: 455 - RNA, 22 nt, Homo Sapiens
cucuagagggaagcacuuucuc

SEQ ID NO: 456 - RNA, 22 nt, Homo Sapiens
aaagugcauccuuuuagagugu

SEQ ID NO: 457 - RNA, 22 nt, Homo Sapiens
cucuagagggaagcgcuuucug

SEQ ID NO: 458 - RNA, 22 nt, Homo Sapiens
aaagugcauccuuuuagagguu

SEQ ID NO: 459 - RNA, 22 nt, Homo Sapiens
cucuagagggaagcgcuuucug

SEQ ID NO: 460 - RNA, 22 nt, Homo Sapiens
aaagugcaucuuuuuagaggau

SEQ ID NO: 461 - RNA, 22 nt, Homo Sapiens
cucuagagggaagcgcuuucug

SEQ ID NO: 462 - RNA, 22 nt, Homo Sapiens
caaagugccucccuuuagagug

SEQ ID NO: 463 - RNA, 22 nt, Homo Sapiens
aagugccuccuuuuagaguguu

SEQ ID NO: 464 - RNA, 22 nt, Homo Sapiens
uucuccaaaagggagcacuuuc

SEQ ID NO: 465 - RNA, 22 nt, Homo Sapiens
aaagugcuucccuuuggacugu

SEQ ID NO: 466 - RNA, 21 nt, Homo Sapiens
cuccagagggaaguacuuucu

SEQ ID NO: 467 - RNA, 21 nt, Homo Sapiens
aaagugcuuccuuuuagaggg

SEQ ID NO: 468 - RNA, 22 nt, Homo Sapiens
aaagugcuuccuuuuagagggu

SEQ ID NO: 469 - RNA, 22 nt, Homo Sapiens
cucuagagggaagcacuuucug

SEQ ID NO: 470 - RNA, 22 nt, Homo Sapiens
aaagugcuucucuuuggugggu

SEQ ID NO: 471 - RNA, 20 nt, Homo Sapiens
cuacaaagggaagcccuuuc

SEQ ID NO: 472 - RNA, 21 nt, Homo Sapiens
aaagugcuuccuuuuugaggg

SEQ ID NO: 473 - RNA, 22 nt, Homo Sapiens
aagugcuuccuuuuagaggguu

SEQ ID NO: 474 - RNA, 24 nt, Homo Sapiens
acaaagugcuucccuuuagagugu

SEQ ID NO: 475 - RNA, 22 nt, Homo Sapiens
acaaagugcuucccuuuagagu

SEQ ID NO: 476 - RNA, 22 nt, Homo Sapiens
aacgcacuucccuuuagagugu

SEQ ID NO: 477 - RNA, 22 nt, Homo Sapiens
aaaaugguucccuuuagagugu

SEQ ID NO: 478 - RNA, 22 nt, Homo Sapiens
cucuagagggaagcgcuuucug

SEQ ID NO: 479 - RNA, 23 nt, Homo Sapiens
gaacgcgcuucccuauagagggu

SEQ ID NO: 480 - RNA, 22 nt, Homo Sapiens
cucuagagggaagcgcuuucug

SEQ ID NO: 481 - RNA, 21 nt, Homo Sapiens
gaaggcgcuucccuuuggagu

SEQ ID NO: 482 - RNA, 22 nt, Homo Sapiens
cuacaaagggaagcacuuucuc

SEQ ID NO: 483 - RNA, 22 nt, Homo Sapiens
gaaggcgcuucccuuuagagcg

SEQ ID NO: 484 - RNA, 21 nt, Homo Sapiens
cuccagagggaugcacuuucu

SEQ ID NO: 485 - RNA, 22 nt, Homo Sapiens
cucuagagggaagcacuuucug

SEQ ID NO: 486 - RNA, 23 nt, Homo Sapiens
cucuugagggaagcacuuucugu

SEQ ID NO: 487 - RNA, 22 nt, Homo Sapiens
gaaagugcuuccuuuuagaggc

SEQ ID NO: 488 - RNA, 20 nt, Homo Sapiens
cugcaaagggaagcccuuuc

SEQ ID NO: 489 - RNA, 22 nt, Homo Sapiens
ccucccacacccaaggcuugca

SEQ ID NO: 490 - RNA, 22 nt, Homo Sapiens
caugccuugaguguaggaccgu

SEQ ID NO: 491 - RNA, 22 nt, Homo Sapiens
ggagaaauuauccuuggugugu

SEQ ID NO: 492 - RNA, 22 nt, Homo Sapiens
uggugggcacagaaucuggacu

SEQ ID NO: 493 - RNA, 25 nt, Homo Sapiens
aaaggauucugcugucggucccacu

SEQ ID NO: 494 - RNA, 22 nt, Homo Sapiens
ugugacagauugauaacugaaa

SEQ ID NO: 495 - RNA, 23 nt, Homo Sapiens
ucggggaucaucaugucacgaga

SEQ ID NO: 496 - RNA, 22 nt, Homo Sapiens
aaacauucgcggugcacuucuu

SEQ ID NO: 497 - RNA, 22 nt, Homo Sapiens
auucugcauuuuuagcaaguuc

SEQ ID NO: 498 - RNA, 22 nt, Homo Sapiens
ucagcaaacauuuauugugugc

SEQ ID NO: 499 - RNA, 22 nt, Homo Sapiens
ucaguaaauguuuauuagauga

SEQ ID NO: 500 - RNA, 22 nt, Homo Sapiens
caaaacuggcaauuacuuuugc

SEQ ID NO: 501 - RNA, 22 nt, Homo Sapiens
aaaaguaauugcgaguuuuacc

SEQ ID NO: 502 - RNA, 22 nt, Homo Sapiens
caagaaccucaguugcuuuugu

SEQ ID NO: 503 - RNA, 22 nt, Homo Sapiens
aaaaguaauugugguuuuggcc

SEQ ID NO: 504 - RNA, 22 nt, Homo Sapiens
caaaaaucucaauuacuuuugc

SEQ ID NO: 505 - RNA, 22 nt, Homo Sapiens
aaaaguaauugcgguuuuugcc

SEQ ID NO: 506 - RNA, 22 nt, Homo Sapiens
caaaaaccacaguuucuuuugc

SEQ ID NO: 507 - RNA, 22 nt, Homo Sapiens
aaaaguaauugugguuuuugcc

SEQ ID NO: 508 - RNA, 21 nt, Homo Sapiens
ugacaacuauggaugagcucu

SEQ ID NO: 509 - RNA, 23 nt, Homo Sapiens
agugccugagggaguaagagccc

SEQ ID NO: 510 - RNA, 22 nt, Homo Sapiens
ugucuuacucccucaggcacau

SEQ ID NO: 511 - RNA, 21 nt, Homo Sapiens
gcgacccacucuugguuucca

SEQ ID NO: 512 - RNA, 21 nt, Homo Sapiens
gcgacccauacuugguuucag

SEQ ID NO: 513 - RNA, 22 nt, Homo Sapiens
gaaaucaagcgugggugagacc

SEQ ID NO: 514 - RNA, 21 nt, Homo Sapiens
aacaggugacugguuagacaa

SEQ ID NO: 515 - RNA, 21 nt, Homo Sapiens
aaaacggugagauuuuguuuu

SEQ ID NO: 516 - RNA, 21 nt, Homo Sapiens
gcuaguccugacucagccagu

SEQ ID NO: 517 - RNA, 21 nt, Homo Sapiens
aggguaagcugaaccucugau

SEQ ID NO: 518 - RNA, 22 nt, Homo Sapiens
auauuaccauuagcucaucuuu

SEQ ID NO: 519 - RNA, 22 nt, Homo Sapiens
gaugagcucauuguaauaugag

SEQ ID NO: 520 - RNA, 23 nt, Homo Sapiens
guuugcacgggugggccuugucu

SEQ ID NO: 521 - RNA, 19 nt, Homo Sapiens
ugagcugcuguaccaaaau

SEQ ID NO: 522 - RNA, 21 nt, Homo Sapiens
uaaaguaaauaugcaccaaaa

SEQ ID NO: 523 - RNA, 20 nt, Homo Sapiens
gcgugcgccggccggccgcc

SEQ ID NO: 524 - RNA, 22 nt, Homo Sapiens
caaaguuuaagauccuugaagu

SEQ ID NO: 525 - RNA, 20 nt, Homo Sapiens
aaaguagcuguaccauuugc

SEQ ID NO: 526 - RNA, 19 nt, Homo Sapiens
agguugacauacguuuccc

SEQ ID NO: 527 - RNA, 19 nt, Homo Sapiens
aggcacggugucagcaggc

SEQ ID NO: 528 - RNA, 22 nt, Homo Sapiens
ggcuggcucgcgaugucuguuu

SEQ ID NO: 529 - RNA, 19 nt, Homo Sapiens
gggcgccugugaucccaac

SEQ ID NO: 530 - RNA, 23 nt, Homo Sapiens
aguauguucuuccaggacagaac

SEQ ID NO: 531 - RNA, 20 nt, Homo Sapiens
auguauaaauguauacacac

SEQ ID NO: 532 - RNA, 21 nt, Homo Sapiens
aguuaaugaauccuggaaagu

SEQ ID NO: 533 - RNA, 22 nt, Homo Sapiens
cgaaaacagcaauuaccuuugc

SEQ ID NO: 534 - RNA, 21 nt, Homo Sapiens
ugaguuggccaucugagugag

SEQ ID NO: 535 - RNA, 20 nt, Homo Sapiens
guccgcucggcgguggccca

SEQ ID NO: 536 - RNA, 24 nt, Homo Sapiens
cugaagugauguguaacugaucag

SEQ ID NO: 537 - RNA, 22 nt, Homo Sapiens
cacgcucaugcacacacccaca

SEQ ID NO: 538 - RNA, 23 nt, Homo Sapiens
ugagugugugugugugagugugu

SEQ ID NO: 539 - RNA, 19 nt, Homo Sapiens
gagccaguuggacaggagc

SEQ ID NO: 540 - RNA, 22 nt, Homo Sapiens
aagauguggaaaaauuggaauc

SEQ ID NO: 541 - RNA, 22 nt, Homo Sapiens
auucuaauuucuccacgucuuu

SEQ ID NO: 542 - RNA, 21 nt, Homo Sapiens
uagauaaaauauugguaccug

SEQ ID NO: 543 - RNA, 21 nt, Homo Sapiens
cuucuugugcucuaggauugu

SEQ ID NO: 544 - RNA, 23 nt, Homo Sapiens
uucauuugguauaaaccgcgauu

SEQ ID NO: 545 - RNA, 22 nt, Homo Sapiens
uugagaaugaugaaucauuagg

SEQ ID NO: 546 - RNA, 21 nt, Homo Sapiens
ucuuguguucucuagaucagu

SEQ ID NO: 547 - RNA, 22 nt, Homo Sapiens
uaacugguugaacaacugaacc

SEQ ID NO: 548 - RNA, 23 nt, Homo Sapiens
uuacaguuguucaaccaguuacu

SEQ ID NO: 549 - RNA, 21 nt, Homo Sapiens
caaagaggaaggucccauuac

SEQ ID NO: 550 - RNA, 22 nt, Homo Sapiens
uuaugguuugccugggacugag

SEQ ID NO: 551 - RNA, 19 nt, Homo Sapiens
ugggcguaucuguaugcua

SEQ ID NO: 552 - RNA, 22 nt, Homo Sapiens
uaugcauuguauuuuuaggucc

SEQ ID NO: 553 - RNA, 21 nt, Homo Sapiens
uuuccauaggugaugagucac

SEQ ID NO: 554 - RNA, 21 nt, Homo Sapiens
uuggccacaauggguuagaac

SEQ ID NO: 555 - RNA, 22 nt, Homo Sapiens
ugagaaccacgucugcucugag

SEQ ID NO: 556 - RNA, 24 nt, Homo Sapiens
ucagaacaaaugccgguucccaga

SEQ ID NO: 557 - RNA, 21 nt, Homo Sapiens
uaauuuuauguauaagcuagu

SEQ ID NO: 558 - RNA, 22 nt, Homo Sapiens
gagcuuauucauaaaagugcag

SEQ ID NO: 559 - RNA, 20 nt, Homo Sapiens
agaccauggguucucauugu

SEQ ID NO: 560 - RNA, 22 nt, Homo Sapiens
uugugucaauaugcgaugaugu

SEQ ID NO: 561 - RNA, 19 nt, Homo Sapiens
ugucucugcugggguuucu

SEQ ID NO: 562 - RNA, 25 nt, Homo Sapiens
aggcaccagccaggcauugcucagc

SEQ ID NO: 563 - RNA, 21 nt, Homo Sapiens
gaagugugccguggugugucu

SEQ ID NO: 564 - RNA, 21 nt, Homo Sapiens
aagccugcccggcuccucggg

SEQ ID NO: 565 - RNA, 22 nt, Homo Sapiens
ugugucacucgaugaccacugu

SEQ ID NO: 566 - RNA, 22 nt, Homo Sapiens
uacgucaucguugucaucguca

SEQ ID NO: 567 - RNA, 20 nt, Homo Sapiens
guugugucaguuuaucaaac

SEQ ID NO: 568 - RNA, 23 nt, Homo Sapiens
acuuacagacaagagccuugcuc

SEQ ID NO: 569 - RNA, 22 nt, Homo Sapiens
uggucuaggauuguuggaggag

SEQ ID NO: 570 - RNA, 23 nt, Homo Sapiens
gacacgggcgacagcugcggccc

SEQ ID NO: 571 - RNA, 22 nt, Homo Sapiens
cacacacugcaauuacuuuugc

SEQ ID NO: 572 - RNA, 19 nt, Homo Sapiens
aggcugcggaauucaggac

SEQ ID NO: 573 - RNA, 23 nt, Homo Sapiens
uaaaucccauggugccuucuccu

SEQ ID NO: 574 - RNA, 21 nt, Homo Sapiens
aaacuacugaaaaucaaagau

SEQ ID NO: 575 - RNA, 21 nt, Homo Sapiens
guucaaauccagaucuauaac

SEQ ID NO: 576 - RNA, 25 nt, Homo Sapiens
aggggugguguugggacagcuccgu

SEQ ID NO: 577 - RNA, 20 nt, Homo Sapiens
aggguguuucucucaucucu

SEQ ID NO: 578 - RNA, 21 nt, Homo Sapiens
ugagcuaaaugugugcuggga

SEQ ID NO: 579 - RNA, 23 nt, Homo Sapiens
gcgaggaccccucggggucugac

SEQ ID NO: 580 - RNA, 25 nt, Homo Sapiens
gcugggcagggcuucugagcuccuu

SEQ ID NO: 581 - RNA, 20 nt, Homo Sapiens
aggaauguuccuucuuugcc

SEQ ID NO: 582 - RNA, 23 nt, Homo Sapiens
gaacgccuguucuugccaggugg

SEQ ID NO: 583 - RNA, 22 nt, Homo Sapiens
uccgagccugggucucccucuu

SEQ ID NO: 584 - RNA, 22 nt, Homo Sapiens
ggggguccccggugcucggauc

SEQ ID NO: 585 - RNA, 22 nt, Homo Sapiens
agucauuggaggguuugagcag

SEQ ID NO: 586 - RNA, 22 nt, Homo Sapiens
acucaaaacccuucagugacuu

SEQ ID NO: 587 - RNA, 22 nt, Homo Sapiens
agacuucccauuugaagguggc

SEQ ID NO: 588 - RNA, 23 nt, Homo Sapiens
aaacucuacuuguccuucugagu

SEQ ID NO: 589 - RNA, 24 nt, Homo Sapiens
gaccuggacauguuugugcccagu

SEQ ID NO: 590 - RNA, 20 nt, Homo Sapiens
auggagauagauauagaaau

SEQ ID NO: 591 - RNA, 21 nt, Homo Sapiens
ggcuagcaacagcgcuuaccu

SEQ ID NO: 592 - RNA, 21 nt, Homo Sapiens
acagucugcugagguuggagc

SEQ ID NO: 593 - RNA, 23 nt, Homo Sapiens
aucccuugcaggggcuguugggu

SEQ ID NO: 594 - RNA, 21 nt, Homo Sapiens
cacaagguauugguauuaccu

SEQ ID NO: 595 - RNA, 22 nt, Homo Sapiens
uaguaccaguaccuuguguuca

SEQ ID NO: 596 - RNA, 21 nt, Homo Sapiens
agggggaaaguucuauagucc

SEQ ID NO: 597 - RNA, 22 nt, Homo Sapiens
gacuauagaacuuucccccuca

SEQ ID NO: 598 - RNA, 19 nt, Homo Sapiens
agcugucugaaaaugucuu

SEQ ID NO: 599 - RNA, 22 nt, Homo Sapiens
gugagucucuaagaaaagagga

SEQ ID NO: 600 - RNA, 21 nt, Homo Sapiens
ucuaguaagaguggcagucga

SEQ ID NO: 601 - RNA, 22 nt, Homo Sapiens
augcugacauauuuacuagagg

SEQ ID NO: 602 - RNA, 21 nt, Homo Sapiens
uggguuuacguugggagaacu

SEQ ID NO: 603 - RNA, 22 nt, Homo Sapiens
guucucccaacguaagcccagc

SEQ ID NO: 604 - RNA, 22 nt, Homo Sapiens
aguauucuguaccagggaaggu

SEQ ID NO: 605 - RNA, 21 nt, Homo Sapiens
agaccuggcccagaccucagc

SEQ ID NO: 606 - RNA, 19 nt, Homo Sapiens
gugucugcuuccuguggga

SEQ ID NO: 607 - RNA, 23 nt, Homo Sapiens
cuaauaguaucuaccacaauaaa

SEQ ID NO: 608 - RNA, 22 nt, Homo Sapiens
aaccagcaccccaacuuuggac

SEQ ID NO: 609 - RNA, 23 nt, Homo Sapiens
acuugggcacugaaacaaugucc

SEQ ID NO: 610 - RNA, 23 nt, Homo Sapiens
ugugcuugcucgucccgcccgca

SEQ ID NO: 611 - RNA, 24 nt, Homo Sapiens
acugggggcuuucgggcucugcgu

SEQ ID NO: 612 - RNA, 25 nt, Homo Sapiens
agggaucgcgggcggguggcggccu

SEQ ID NO: 613 - RNA, 23 nt, Homo Sapiens
aucgcugcgguugcgagcgcugu

SEQ ID NO: 614 - RNA, 21 nt, Homo Sapiens
augauccaggaaccugccucu

SEQ ID NO: 615 - RNA, 24 nt, Homo Sapiens
aaagacauaggauagagucaccuc

SEQ ID NO: 616 - RNA, 22 nt, Homo Sapiens
gucccucuccaaaugugucuug

SEQ ID NO: 617 - RNA, 22 nt, Homo Sapiens
acuuguaugcuagcucagguag

SEQ ID NO: 618 - RNA, 19 nt, Homo Sapiens
aguguggcuuucuuagagc

SEQ ID NO: 619 - RNA, 19 nt, Homo Sapiens
ucuaggcugguacugcuga

SEQ ID NO: 620 - RNA, 19 nt, Homo Sapiens
aagcagcugccucugaggc

SEQ ID NO: 621 - RNA, 21 nt, Homo Sapiens
guggcugcacucacuuccuuc

SEQ ID NO: 622 - RNA, 19 nt, Homo Sapiens
aagugugcagggcacuggu

SEQ ID NO: 623 - RNA, 22 nt, Homo Sapiens
aaaccuguguuguucaagaguc

SEQ ID NO: 624 - RNA, 21 nt, Homo Sapiens
aggaggcagcgcucucaggac

SEQ ID NO: 625 - RNA, 22 nt, Homo Sapiens
uuuaggauaagcuugacuuuug

SEQ ID NO: 626 - RNA, 21 nt, Homo Sapiens
aauggcgccacuaggguugug

SEQ ID NO: 627 - RNA, 21 nt, Homo Sapiens
guguugaaacaaucucuacug

SEQ ID NO: 628 - RNA, 22 nt, Homo Sapiens
uaugucugcugaccaucaccuu

SEQ ID NO: 629 - RNA, 22 nt, Homo Sapiens
uggugggccgcagaacaugugc

SEQ ID NO: 630 - RNA, 22 nt, Homo Sapiens
auaauacaugguuaaccucuuu

SEQ ID NO: 631 - RNA, 21 nt, Homo Sapiens
aauauuauacagucaaccucu

SEQ ID NO: 632 - RNA, 23 nt, Homo Sapiens
ggcagguucucacccucucuagg

SEQ ID NO: 633 - RNA, 25 nt, Homo Sapiens
ggcggagggaaguagguccguuggu

SEQ ID NO: 634 - RNA, 22 nt, Homo Sapiens
cuugguucagggagggucccca

SEQ ID NO: 635 - RNA, 22 nt, Homo Sapiens
uacccauugcauaucggaguug

SEQ ID NO: 636 - RNA, 24 nt, Homo Sapiens
ugccugggucucuggccugcgcgu

SEQ ID NO: 637 - RNA, 21 nt, Homo Sapiens
ucccacguuguggcccagcag

SEQ ID NO: 638 - RNA, 22 nt, Homo Sapiens
aggcggggcgccgcgggaccgc

SEQ ID NO: 639 - RNA, 20 nt, Homo Sapiens
accaggaggcugaggccccu

SEQ ID NO: 640 - RNA, 23 nt, Homo Sapiens
ugucacucggcucggcccacuac

SEQ ID NO: 641 - RNA, 21 nt, Homo Sapiens
uccgguucucagggcuccacc

SEQ ID NO: 642 - RNA, 23 nt, Homo Sapiens
aggaagcccuggaggggcuggag

SEQ ID NO: 643 - RNA, 23 nt, Homo Sapiens
ugagguugguguacuguguguga

SEQ ID NO: 644 - RNA, 22 nt, Homo Sapiens
gcacugagaugggaguggugua

SEQ ID NO: 645 - RNA, 23 nt, Homo Sapiens
uggugcggagagggcccacagug

SEQ ID NO: 646 - RNA, 23 nt, Homo Sapiens
uggaagacuagugauuuuguugu

SEQ ID NO: 647 - RNA, 23 nt, Homo Sapiens
aaggagcuuacaaucuagcuggg

SEQ ID NO: 648 - RNA, 22 nt, Homo Sapiens
caacuagacugugagcuucuag

SEQ ID NO: 649 - RNA, 22 nt, Homo Sapiens
caacaaaucacagucugccaua

SEQ ID NO: 650 - RNA, 22 nt, Homo Sapiens
caacaaaucccagucuaccuaa

SEQ ID NO: 651 - RNA, 22 nt, Homo Sapiens
ugcggggcuagggcuaacagca

SEQ ID NO: 652 - RNA, 22 nt, Homo Sapiens
cuguugccacuaaccucaaccu

SEQ ID NO: 653 - RNA, 22 nt, Homo Sapiens
uuugugaccugguccacuaacc

SEQ ID NO: 654 - RNA, 20 nt, Homo Sapiens
cggcucugggucugugggga

SEQ ID NO: 655 - RNA, 21 nt, Homo Sapiens
uggaggagaaggaaggugaug

SEQ ID NO: 656 - RNA, 22 nt, Homo Sapiens
acuccagccccacagccucagc

SEQ ID NO: 657 - RNA, 23 nt, Homo Sapiens
ucugcucauaccccaugguuucu

SEQ ID NO: 658 - RNA, 23 nt, Homo Sapiens
ugcaccaugguugucugagcaug

SEQ ID NO: 659 - RNA, 28 nt, Homo Sapiens
ucacaaugcugacacucaaacugcugac

SEQ ID NO: 660 - RNA, 26 nt, Homo Sapiens
guuggaggaugaaaguacggagugau

SEQ ID NO: 661 - RNA, 23 nt, Homo Sapiens
cugggaucuccggggucuugguu

SEQ ID NO: 662 - RNA, 22 nt, Homo Sapiens
ugagaccucuggguucugagcu

SEQ ID NO: 663 - RNA, 23 nt, Homo Sapiens
uccaguaccacgugucagggcca

SEQ ID NO: 664 - RNA, 24 nt, Homo Sapiens
gauugcucugcgugcggaaucgac

SEQ ID NO: 665 - RNA, 23 nt, Homo Sapiens
caguaacaaagauucauccuugu

SEQ ID NO: 666 - RNA, 23 nt, Homo Sapiens
uauucagauuagugccagucaug

SEQ ID NO: 667 - RNA, 21 nt, Homo Sapiens
aagguuacuuguuaguucagg

SEQ ID NO: 668 - RNA, 21 nt, Homo Sapiens
gcaggaacuugugagucuccu

SEQ ID NO: 669 - RNA, 22 nt, Homo Sapiens
cugcccuggcccgagggaccga

SEQ ID NO: 670 - RNA, 21 nt, Homo Sapiens
ccuggaaacacugagguugug

SEQ ID NO: 671 - RNA, 22 nt, Homo Sapiens
uauaccucaguuuuaucaggug

SEQ ID NO: 672 - RNA, 22 nt, Homo Sapiens
uggugguuuacaaaguaauuca

SEQ ID NO: 673 - RNA, 22 nt, Homo Sapiens
uggauuucuuugugaaucacca

SEQ ID NO: 674 - RNA, 20 nt, Homo Sapiens
guagaggagauggcgcaggg

SEQ ID NO: 675 - RNA, 22 nt, Homo Sapiens
uccucuucucccuccucccagg

SEQ ID NO: 676 - RNA, 22 nt, Homo Sapiens
aggcagcgggguguaguggaua

SEQ ID NO: 677 - RNA, 22 nt, Homo Sapiens
uccauuacacuacccugccucu

SEQ ID NO: 678 - RNA, 21 nt, Homo Sapiens
cgcgggugcuuacugacccuu

SEQ ID NO: 679 - RNA, 23 nt, Homo Sapiens
cgggucggaguuagcucaagcgg

SEQ ID NO: 680 - RNA, 22 nt, Homo Sapiens
gugaacgggcgccaucccgagg

SEQ ID NO: 681 - RNA, 21 nt, Homo Sapiens
uacucaaaaagcugucaguca

SEQ ID NO: 682 - RNA, 22 nt, Homo Sapiens
gacugacaccucuuugggugaa

SEQ ID NO: 683 - RNA, 21 nt, Homo Sapiens
uuaauaucggacaaccauugu

SEQ ID NO: 684 - RNA, 21 nt, Homo Sapiens
uacuuggaaaggcaucaguug

SEQ ID NO: 685 - RNA, 22 nt, Homo Sapiens
ugcaacgaaccugagccacuga

SEQ ID NO: 686 - RNA, 22 nt, Homo Sapiens
ugcaacuuaccugagucauuga

SEQ ID NO: 687 - RNA, 21 nt, Homo Sapiens
cacuguguccuuucugcguag

SEQ ID NO: 688 - RNA, 22 nt, Homo Sapiens
cacuggcuccuuucuggguaga

SEQ ID NO: 689 - RNA, 23 nt, Homo Sapiens
ucuuugguuaucuagcuguauga

SEQ ID NO: 690 - RNA, 22 nt, Homo Sapiens
auaaagcuagauaaccgaaagu

SEQ ID NO: 691 - RNA, 20 nt, Homo Sapiens
ggggagcuguggaagcagua

SEQ ID NO: 692 - RNA, 25 nt, Homo Sapiens
cuagugagggacagaaccaggauuc

SEQ ID NO: 693 - RNA, 23 nt, Homo Sapiens
gcagcagagaauaggacuacguc

SEQ ID NO: 694 - RNA, 21 nt, Homo Sapiens
gucagcggaggaaaagaaacu

SEQ ID NO: 695 - RNA, 20 nt, Homo Sapiens
agagucuugugaugucuugc

SEQ ID NO: 696 - RNA, 22 nt, Homo Sapiens
uauugcacuugucccggccugu

SEQ ID NO: 697 - RNA, 23 nt, Homo Sapiens
agguugggaucgguugcaaugcu

SEQ ID NO: 698 - RNA, 22 nt, Homo Sapiens
ggguggggauuuguugcauuac

SEQ ID NO: 699 - RNA, 22 nt, Homo Sapiens
uauugcacucgucccggccucc

SEQ ID NO: 700 - RNA, 22 nt, Homo Sapiens
agggacgggacgcggugcagug

SEQ ID NO: 701 - RNA, 23 nt, Homo Sapiens
caaagugcuguucgugcagguag

SEQ ID NO: 702 - RNA, 22 nt, Homo Sapiens
acugcugagcuagcacuucccg

SEQ ID NO: 703 - RNA, 22 nt, Homo Sapiens
ugugcgcagggagaccucuccc

SEQ ID NO: 704 - RNA, 22 nt, Homo Sapiens
ugucuacuacuggagacacugg

SEQ ID NO: 705 - RNA, 23 nt, Homo Sapiens
ccaguuaccgcuuccgcuaccgc

SEQ ID NO: 706 - RNA, 22 nt, Homo Sapiens
acaguagagggaggaaucgcag

SEQ ID NO: 707 - RNA, 22 nt, Homo Sapiens
auccgcgcucugacucucugcc

SEQ ID NO: 708 - RNA, 22 nt, Homo Sapiens
ugcccuuaaaggugaacccagu

SEQ ID NO: 709 - RNA, 24 nt, Homo Sapiens
uggggagcugaggcucugggggug

SEQ ID NO: 710 - RNA, 21 nt, Homo Sapiens
aaggcagggcccccgcucccc

SEQ ID NO: 711 - RNA, 23 nt, Homo Sapiens
cacccggcugugugcacaugugc

SEQ ID NO: 712 - RNA, 22 nt, Homo Sapiens
ucuucucuguuuuggccaugug

SEQ ID NO: 713 - RNA, 21 nt, Homo Sapiens
cugacuguugccguccuccag

SEQ ID NO: 714 - RNA, 22 nt, Homo Sapiens
aaauuauuguacaucggaugag

SEQ ID NO: 715 - RNA, 22 nt, Homo Sapiens
uucaacggguauuuauugagca

SEQ ID NO: 716 - RNA, 23 nt, Homo Sapiens
uuuggcacuagcacauuuuugcu

SEQ ID NO: 717 - RNA, 22 nt, Homo Sapiens
aaucaugugcagugccaauaug

SEQ ID NO: 718 - RNA, 22 nt, Homo Sapiens
ugagguaguaaguuguauuguu

SEQ ID NO: 719 - RNA, 22 nt, Homo Sapiens
aacccguagauccgaucuugug

SEQ ID NO: 720 - RNA, 22 nt, Homo Sapiens
caagcucgcuucuaugggucug

SEQ ID NO: 721 - RNA, 22 nt, Homo Sapiens
cacccguagaaccgaccuugcg

SEQ ID NO: 722 - RNA, 22 nt, Homo Sapiens
caagcucgugucuguggguccg

SEQ ID NO: 723 - RNA, 20 nt, Herpes Simplex
uggcggcccggcccggggcc